(19) United States
(12) Patent Application Publication
(10) Pub. No.: US 2025/0345440 A1
(43) Pub. Date: Nov. 13, 2025

(54) METAL-ORGANIC FRAMEWORK MATERIALS AND USES OF THE METAL-ORGANIC FRAMEWORK MATERIALS

(71) Applicant: MOF Science, LLC, Fort Lauderdale, FL (US)

(72) Inventors: Marcelo Ferro, Miramar, FL (US); Richard Todd Ullom, Fort Lauderdale, FL (US)

(21) Appl. No.: 19/204,432

(22) Filed: May 9, 2025

Related U.S. Application Data
(60) Provisional application No. 63/645,245, filed on May 10, 2024.
(60) Provisional application No. 63/645,249, filed on May 10, 2024.
(60) Provisional application No. 63/645,252, filed on May 10, 2024.

(51) Int. Cl. A61K 47/54; A61K 47/69

(52) U.S. Cl. CPC A61K 47/547; A61K 47/6949

(57) ABSTRACT

Embodiments of the invention provide modified metal-organic frameworks (MOF), materials and compositions comprising the modified metal-organic frameworks (MOF) and uses of the materials and compositions comprising modified MOFs. The modified MOFs may include a functionalizing constituent that provides enhanced functionality, such as transporting molecules across biological membranes. Embodiments of the modified MOF may comprise a magnesium-gallate (Mg-GA) network structure. The Mg-GA MOF may also comprise phosphate-functionalized polyethylene glycol, which comprises PEGylates (polyethylene glycol) with phosphate groups.

### CROSS REFERENCE TO RELATED APPLICATIONS

In accordance with 37 C.F.R. 1.76, a claim of priority is included in an Application Data Sheet filed concurrently herewith. Accordingly, the present invention claims priority to U.S. Provisional Patent Application No. 63/645,245, entitled “METAL-ORGANIC FRAMEWORK MATERIALS AND USES OF THE METAL-ORGANIC FRAMEWORK MATERIALS”, filed on May 10, 2024, to U.S. Provisional Patent Application No. 63/645,249, entitled “NICOTINAMIDE ADENINE DINUCLEOTIDE (NAD)-LOADED IN MAGNESIUM GALLATE MOF FOR USE IN METABOLIC SYNDROME AND AGING PROCESSES”, filed on May 10, 2024, and to U.S. Provisional Patent Application No. 63/645,252, entitled “SUPEROXIDE DISMUTASE-LOADED IN MAGNESIUM GALLATE MOF FOR USE IN METABOLIC SYNDROME AND AGING PROCESSES” filed on May 10, 2024. The contents of the above referenced applications are incorporated herein by reference in their entirety.

### FIELD OF THE INVENTION

The invention relates to metal-organic frameworks (MOF), MOF materials or compositions, and methods of using the MOF materials or compositions; to modified MOF, modified MOF materials or compositions, and methods of using the modified MOF materials or compositions; to functionalized modified magnesium gallate MOF, functionalized modified magnesium gallate MOF materials or compositions, and the use of modified magnesium gallate MOF materials or compositions as a vehicle to transport molecules across biological membranes; and more particularly to loaded, functionalized modified magnesium gallate MOF, functionalized modified magnesium gallate MOF materials or compositions, and the use of loaded, modified magnesium gallate MOF materials or compositions as a vehicle to transport molecules across biological membranes.

### BACKGROUND OF THE INVENTION

Biological membranes consist of a bilayer of lipid molecules, referred to as a phospholipid bilayer. In addition to the various types of lipids that occur in biological membranes, membrane proteins and sugars are also key components of the structure. Membrane proteins play a vital role in biological membranes by maintaining structural integrity, organization, and flow of material across membranes. Sugars are found only on one side of the bilayer and are linked by covalent bonds to some lipids and proteins (Essays Biochem. 2015 Nov. 15; 59: 43-69. Watson H).

There are three types of lipids found in biological membranes: phospholipids, glycolipids and sterols. Phospholipids consist of two fatty acid chains linked to glycerol and a phosphate group. Glycerol-containing phospholipids are called glycerophospholipids (Encyclopedia of Behavioral Neuroscience, 2nd edition, 2022, Pages 372-382. Johnson and Johnson). An example of a glycerophospholipid commonly found in biological membranes is phosphatidylcholine, which has a choline molecule linked to the phosphate group. Serine and ethanolamine can replace choline at this position, and these lipids are called phosphatidylserine and phosphatidylethanolamine, respectively (Molecular Biology of the Cell. 4th edition. Garland Science; 2002. Albert et al.). Glycolipids are lipids with a carbohydrate (monosaccharide or oligosaccharide) attached by a glycoside bond. Sterols are absent from most bacterial membranes, but are an important component of animal membranes (normally cholesterol). Cholesterol has a structure quite different from that of phospholipids and glycolipids. Cholesterol consists of a hydroxyl group (which is the hydrophilic “head” region), a four-ring steroid backbone, and a short hydrocarbon side chain (Essays Biochem. 2015 Nov. 15; 59: 43-69. Watson H.).

All membrane lipids are amphipathic, containing a hydrophilic region (which attracts water) and a hydrophobic region (which repels water). Thus, the most favorable environment for the hydrophilic head is aqueous, while the hydrophobic tail is stable in more a lipid environment (Molecular Biology of the Cell. 4th edition. Garland Science; 2002. Albert et al). The amphipathic nature of membrane lipids means that they naturally form bilayers in which the hydrophilic heads point outward toward the aqueous environment and the hydrophobic tails point inward toward each other. When placed in water, membrane lipids spontaneously form liposomes, which are spheres, formed by a bilayer with water inside and outside, resembling a small cell. This is the classic configuration for lipids, as it means that all hydrophilic heads are in contact with water and all hydrophobic tails are in a lipid environment (Essays Biochem. 2015 Nov. 15; 59: 43-69. Watson H).

Metal-organic structures (MOFs) are hybrid (organic-inorganic) crystalline porous materials consisting of a regular matrix of positively charged metal ions surrounded by “binder” organic molecules. MOFs can form one, two, or three-dimensional structures. They are a subclass of coordination polymers, with the special feature that they are often porous. MOF-based systems size can be controlled, facilitating drug uptake, their toxicity can be minimized using biocompatible ligands and metals, and they can degrade in vivo to clearable components (Journal of Drug Delivery Science and Technology Volume 81, March 2023, 104249, Ahmadi et al. 2021). The characteristics, such as the nontoxic effects of MOFs, directed and stimulus-based delivery systems, multiple drug-loaded properties, and continuous release, have enriched the use of MOFs in drug delivery, biocompatibility, and biodegradability in the last decade (Int J Mol Sci. 2022 April; 23(8): 4458. Maranescu & Visa).

Diverse drug release mechanisms have endowed MOFs with different functions. Currently, MOFs are increasingly used for target drug delivery by combining the MOFs with other materials to form complexes that exhibit multi-stimulus responses to drug release and significantly improved drug targeting ability (RSC Adv. 2021 Jan. 14; 11(6): 3241-3263. Yang et al.). Compared to traditional carrier materials such as liposomes, polymer nanoparticles, mesoporous silica, and inorganic nanomaterials, MOF materials as drug carriers have higher carrying efficiency due to their unique skeleton structure (Pharmaceutics. 2023 September; 15(9): 2309. Xu et al.). Compared with inorganic nanomaterials, MOFs show better biocompatibility and less biological toxicity and can be catabolized more easily by organisms. Due to the diversity and easy functional modification of MOFs, responsive MOF carriers can be designed to achieve responsive release of cargo molecules, especially biological macromolecules, without destroying their biological activity (RSC Adv. 2021 Jan. 14; 11(6): 3241-3263. Yang et al.).

Various parameters such as pH value, optimal buffer, nanoparticle size, and surface adaptation are very important, in addition to appropriate analytical approaches and methodologies to control MOF stability (Int J Mol Sci. 2022 April; 23(8): 4458. Maranescu & Visa). The chemical stability of MOFs relies on pH, time and temperature. Several researchers have combined their efforts to explain the stability of MOF in acids and bases.

Metabolic syndrome is characterized by a group of metabolic risk factors in the same person (Aging Dis. 2015 March; 6(2): 109-120. Bonomini F. et al). One of the defects in metabolic syndrome, and its associated diseases, is excess cellular oxidative stress (reactive oxygen and nitrogen species, ROS/RNS) and oxidative damage to mitochondrial components, resulting in reduced efficiency of the electron transport chain (J Cell Biochem. 2007 Apr. 15;100(6):1352-69. Nicolson G.L.).

Insulin resistance co-exists in varying degrees with a variety of other key risk factors, including dyslipidemia, hypertension, and vascular inflammation, that contribute to poor cardiovascular outcomes of individuals with type 2 diabetes and metabolic syndrome, suggesting that insulin resistance is a physiological compensation to inappropriate oxidative metabolism that induces a metabolic inflammatory response during aging (Biochem Pharmacol. 2006 Jul. 14; 72(2):125-31.; Epub 2006 Feb. 10. Colca J.R.). Thus, the inflammatory response related to fat accumulation may influence cardiovascular risk through its involvement not only in body weight homeostasis, but also in coagulation, fibrinolysis, endothelial dysfunction, and atherosclerosis (Aging Dis. 2015 March; 6(2): 109-120. Bonomini F. et al.). Moreover, oxidative stress may be a mechanistic link between several components of MS and cardiovascular disease (CVD) through its role in inflammation and its ability to disrupt insulin signaling. However, the pathology linked to MS that has been growing the most alarmingly in recent years is non-alcoholic and non-drug liver cirrhosis (Lancet. 2014 May 17; 383(9930):1749-61. Tsochatzis E.A.).

Inflammatory processes begin through a protein complex called nuclear factor kappa-beta (NF-κβ), which induces the expression of various pro-inflammatory genes, including those encoding cytokines and chemokines, and also participates in inflammation regulation. NF-κβ is a key transcriptional regulator of the inflammatory response and plays an essential role in regulating inflammatory signaling pathways in the liver (Signal Transduct Target Ther. 2017; 2: 17023. Liu et al.). First, NF-κβ is activated in virtually every chronic liver disease, including alcoholic liver disease, nonalcoholic fatty liver disease (NAFLD), viral hepatitis, and biliary liver disease. Second, NF-κβ regulates multiple essential functions in hepatocytes, Kupffer cells and hepatic stellate cells (HSCs) as outlined below. Third, genetic inactivation of different NF-κβ signaling components results in liver phenotypes that include spontaneous injury, fibrosis and carcinogenesis, suggesting that NF-κβ makes an essential contribution to liver homeostasis and wound-healing processes (Nat Rev Gastroenterol Hepatol. 2011 February; 8(2): 108-118. Luedde T.).

The central role of NF-κβ in immunological processes and its apparent involvement in several diseases has been reported in several scientific studies (Annu Rev Immunol. 1996; 14:649-83. Baldwin A.S. Jr.). Studies have also reported that the different factors that promote premature aging or delay this process converge in signaling through the NF-κβ pathway (Aging Dis. 2011; 2:449-465. Tilstra et al.). This can be explained by the special characteristics of this protein complex found in practically all animals, especially humans. NF-κβ is involved in cellular responses to different stimuli, such as genotoxic stress, free radicals, ultraviolet irradiation and inflammation.

NF-κβ is known as a fundamental mediator of inflammatory responses that regulates multiple aspects of innate and adaptive immunity (Oncogene. 2006; 25:6758-6780. Hayden et al.). This function of NF-κβ is crucial for aging since one of the main changes that occur during the aging process is the dysregulation of the immune response, which leads to a chronic systemic inflammatory state, generally observed in age-related diseases. (Syst. Biol. Med. 2017; 9:e1370. Munn LL.). Therefore, its role in inflammation, together with previous findings showing that pro-aging stimuli activate NF-κβ signaling, while those with an anti-aging effect inhibit it and, more importantly, that inhibition of NF-κβ activity can delay, and even reverse, the manifestations of aging in human and mouse aging models, making NF-κβ a key protein complex that acts as a driver of aging and should be considered as a potential therapeutic target to prevent premature aging and age-related diseases, including cancer (Aging Dis. 2011; 2:449-465. Tilstra et al., J. Clin. Immunol. 2009; 29:397-405. Salminen & Kaarniranta; Int. Rev. Cell Mol. Biol. 2016; 326:133-174. Osorio et al.). However, NF-κβ may have decreased metabolic activity through the peroxisome proliferator-activated receptors alfa (PPAR-α), a ligand-activated transcriptional factor that belongs to the family of nuclear receptors (Pharm Res. 2004 September; 21(9):1531-8; 2(4): 236-240. Van Raalte et al.).

PPAR-α and β regulate the expression of genes involved in fatty acid beta-oxidation and are a major regulator of energy homeostasis. (Pharm Res. 2004 September; 21(9):1531-8; 2(4): 236-240. Van Raalte et al; Crit Rev Biochem Mol Biol. 2016; 51(1):7-14. Points et al; Endocr Rev. 2018 Oct. 1; 39(5):760-802. Bougarne N.). The PPAR-α and β are ligand-activated receptors with distinct physiological functions in regulating lipid and glucose metabolism, as well as inflammatory response. PPAR-α and β activation allows a coordinated up-regulation not only in numerous fatty acid oxidation (FAO) enzymes but also in significant increases in proteins that participate in mitochondrial biogenesis (Nat Commun. 2019 Apr. 5; 10(1):1566. Iershov A.). Interestingly, recent studies have shown that PPAR-α can be activated by a specific agonist called gallic acid, a phenolic acid present in several plants (Phytomedicine. 2023 January:109:154589. Zhang et al.).

Phenolic acids are an important and abundant subgroup of phenolic compounds with the basic chemical structure of C6-C1 (hydroxybenzoic acids) or C6-C3 (hydroxycinnamic acids), consisting of a phenolic ring and a carboxyl substituent (Iran J Basic Med Sci. 2019 March; 22(3): 225-237. Kahkeshani et al.). The shikimic acid or phenylpropanoid pathway of plant metabolism usually regulates the biosynthesis of phenolic acids. In some cases, phenolic acids are the precursor of other important phytochemicals, such as tannins, coumarins, benzoquinones, and naphthoquinones. Several acids, such as caffeic acid, ferulic acid, vanillic acid, salicylic acid, and gallic acid, are the most common members of phenolic acids (Bioorg Chem. 2016; 64:74-84. Siah et al.).

Endogenous antioxidant defense systems are constituted to reduce reactive oxygen species (ROS). Antioxidant enzymes, including superoxide dismutase (SOD), glutathione peroxidase (GPX), and catalase (CAT), can catalyze the degradation of ROS, while other non-enzymatic antioxidants, including glutathione (GSH), polyamine, and bilirubin, can directly capture and eliminate free radicals, both resulting in the elimination and reduction of cellular deficiencies (Molecules. 2021 December; 26(23): 7115. Xu et al.). External antioxidants, such as vitamin C, vitamin E, carotenoids, and various phenylpropanoid derivatives, have been reported to have the potential to improve antioxidant defense systems. Gallic acid (GA), natural from edible plants, has been applied in nutraceutical products as an antioxidant and regulator of immunity against infections. GA's multifaceted health functions can be attributed primarily to its free radical scavenging ability that helps prevent or alleviate oxidative stress, which is highly involved in metabolic syndrome (Food Funct. 2018; 9:6096-6115. Chhikara et al.).

Although gallic acid exhibits antioxidant properties, it can also exhibit pro-oxidative activities in certain situations. The biological activities of gallic acid may depend on its behavior as an antioxidant or pro-oxidant, depending on tissue physiology. Many studies have proved that its potent free radical scavenging antioxidant activities are involved with ferric reducing antioxidant power (FRAP) and 2,2-diphenyl-1-picrylhydrazyl (DPPH) or oxygen radical absorbance capacity (ORAC) (Molecules. 2018; 23:695. Velderrain-Rodriguez et al.). Because gallic acid has the phenolic hydroxyl group, the hydrogen donor can react with ROS or reactive nitrogen species (RNS) to block the overproduction of damaged free radicals, including peroxyl radicals, hydroxyl and superoxide radicals, and peroxynitrite radicals, preventing the tyrosine nitration.

Gallic acid also influences the mechanism related to the regulation of the operating system, which is associated with the biosynthesis of the enzyme glutathione peroxidase as well as the synthesis of glutathione (Molecules. 2021 December; 26(23): 7115. Xu et al.).

Gallic acid is known to have radioprotective properties against cell damage caused by the adverse side effects of cancer radiotherapy (Phytomedicine. 2018; 47:192-200. Fischer et al.). Administration of gallic acid reduced γ-radiation-induced cellular DNA damage in blood leukocytes, bone marrow cells, and splenocytes of whole-body irradiated mice. The radiation-induced decrease in glutathione peroxidase GPx and GSH levels was restored by the supplementation of gallic acid in various tissues of irradiated mice, with the inhibition of the peroxidation of membrane lipids, consequently leading to lower weight loss and mortality after γ irradiation (BioMed Res. Int. 2013:953079. Nair and Nair.).

Several molecular mechanisms are involved in the administration of gallic acid, among them the modulation of proapoptotic genes (Bax and Bad) and antiapoptotic genes (B-cell lymphoma-extra large (Bcl-xL) and Bcl-2) proteins exhibiting antitumor properties (Pharmacol. 2013; 35:473-485. Verma et al.); potent inhibition of cancer cell invasion and metastasis by downregulation of algogenic substances such as matrix metalloproteinase −2/9 (MMP−2/9) via the anti-inflammatory response (Mol. Nutr. Food Res. 2012; 56:1398-1412. Chen and Chang); inhibition of MMP−2/9 in the suppression of the NF-κβ signaling pathway in gastric adenocarcinoma cell metastasis and the cytoskeletal reorganization (Food Chem. Toxicol. 2010; 48:2508-2516. Ho et al.); the restoration of the antioxidant and inflammatory status to normal levels via nuclear factor erythroid 2-related factor 2 (Nrf2) activation (Free Radic. Res. 2019; 53:210-225. Radan et al.); prevent the induction of TNF-α, lipopolysaccharides (LPS), IL-6, and interferon-α (IFN-α) expression (Molecules. 2021 December; 26(23):7115. Xu et al.); improved the oxidative and inflammatory status that promoted the recovery of the neuronal morphology in the hippocampus (Synapse. 2020; 75:22186. Dias et al.); exerts anti-inflammatory effects against metabolic disorders such as insulin resistance, dyslipidemia, and obesity (Nutr. Res. 2020; 73:58-66. Tanaka et al.); and suppresses the activation of the p65-NF-κβ and IL-6/STAT3 pathways in adipose and decreases adipogenesis by inhibiting the expression of monocyte chemoattractant protein-1 (MCP-1) and increasing that of adiponectin and peroxisome proliferator-activated receptor-γ (PPAR-γ) (Molecules. 2021 December; 26(23):7115. Xu et al.). Thus, gallic acid suppresses adipocyte hypertrophy and inflammation caused by the interaction between adipocytes and macrophages, thereby improving metabolic disorders such as insulin resistance and dyslipidemia (Nutr. Res. 2020; 73:58-66. Tanaka et al.).

Gallic acid also has been reported to be a potential activator of sirtuin 1 (SIRT1) and peroxisome proliferator-activated receptor gamma coactivator-1 alpha (PGC-1α). When activated, PGC-1a has a crucial role in mitochondrial biogenesis (Cell. 1998; 92:829-839. Puigserver et al.), as well as the mitochondrial nicotinamide adenine dinucleotide (NAD), an essential cofactor that regulates metabolic function (Metab. 2011; 14:528-536. Yoshimo et al.). As mitochondria play vital roles including the generation of ATP, regulation of cellular metabolism and cell survival, concentrations of NAD are fundamental to metabolism. A decrease in mitochondrial biogenesis and NAD+ is a hallmark of metabolic diseases, and PGC-1α orchestrates mitochondrial biogenesis and is involved in the mitochondrial NAD+ pool (Lipidol. 2009; 20:98-105. et al. Canto et al.; PLoS ONE. 2011;6:e19194. Braidy et al.; Biochim. Biophys. Acta. 2010; 1797:1028-1033. Zorzano et al.).

The NAD+ pool is important for cellular physiological and metabolic functions for cellular integrity. However, metabolic diseases, such as insulin resistance as well as diabetes, increase NAD+ consumption, demonstrating that lower cellular NAD+ levels are linked to both metabolic diseases and aging (Metab. 2011; 14:528-536. Yoshimo et al.; Exp. Cell Res. 2018; 373:112-118. Waldman et al.). In this context, SIRT1, which consumes NAD+ for cellular metabolic function, is downregulated in several cells and tissues, including myotubes, peripheral blood mononuclear cells, human skeletal muscle and adipose tissue, in states of insulin resistance (Diabetes. 2010; 59:1006-1015. de Kreutzenberg et al.). The decline in NAD+ leads to a drop in SRIT1 expression, accelerating cellular aging processes.

Several studies have shown that SIRT1 regulates glucose homeostasis by regulating insulin secretion and protecting beta (β) cells in the pancreas (Cell Metab. 2005; 2:105-117. Moninyhan et al.; Diabetes. 2010; 59:1006-1015. de Kreutzenberg et al.). Likewise, other studies pointed to improved mitochondrial biogenesis and glucose uptake in skeletal muscle (Med. (Maywood) 2015; 240:557-565. Zhang et al.), promoting improved oxidation of both glucose and fat in the liver (Proc. Natl. Acad. Sci. USA. 2007; 104:12861-12866. Rodger et al.). Overexpression of β-cell-specific SIRT1 in mice improves insulin secretion and glucose tolerance in response to glucose (Cell Metab. 2005; 2:105-117. Moninyhan et al.). Age-related downregulation of SIRT1 activity due to lack of systemic NAD+ biosynthesis results in a decrease in β-cell insulin secretion in response to glucose; however, treatment with nicotinamide mononucleotide (NMN), which is a derivative of niacin and an intermediate in NAD+ biosynthesis in the salvage pathway, restores insulin secretion and improves glucose tolerance in aged mice with specific SIRT1 overexpression for β cells. Therefore, SIRTs regulate glucose-lipid metabolism and mitochondrial biogenesis via PGC-1α (Cell Metab. 2005; 2:105-117. Moninyhan et al.; Exp. Biol. Med. (Maywood) 2015; 240:557-565. Zhang et al.; N. Engl. J. Med. 2011; 364:2235-2244. Guarente et al.). Therefore, the administration of NAD+ may be one of the strategies to improve metabolic dysfunction via SIRTs—PGC-1α, increasing metabolic capacity, including in aging.

Another mechanism of inflammatory and antioxidant protection in cells is the improvement of enzymatic activity that removes ROS, mainly superoxide dismutase (SOD), which is the most important enzyme in controlling ROS production in cells. Usually, cellular problems start with ROS formations. These formations occur through cellular respiration within the mitochondria and in the cell membrane through immunoinflammatory responses. At the mitochondrial level, when electrons migrate from hydrogen to oxygen in the respiratory chain to synthesize ATP, around 2% of the oxygen in this process becomes reactive, forming superoxide (O2—). Because O2— is very reactive, SOD-1 and 2 converts O2— to hydrogen hydroxide (H2O2). Similarly, H2O2 is also toxic to cells and must be converted to H2O by GSH (Principle of Biochemistry, 7th ed January 2017. Nelson D.; Cox M.). Although oxidative stress was defined originally as a balance between oxidants and antioxidant systems, an equilibrium among antioxidant strategies is needed to avoid the generation of oxidants and ROS. Likewise, the excessive production of ROS by NADPH oxidase arising from reactions occurring in the cell membrane needs to be controlled by SOD 1. NADPH oxidase catalyzes the transfer of electrons from NADPH to molecular oxygen to produce ROS. This mechanism distinguishes NADPH oxidases from other oxidases in which ROS production occurs as a byproduct of another oxidative reaction beyond the electron respiratory chain (J Am Soc Nephrol. 2013 October; 24(10): 1512-1518. Sedeek et al.).

SOD supplementation can trigger endogenous antioxidant machinery for the neutralization of excess free radicals and be used in a variety of pathological situations. A systematic review carried out by Rosa et al. (2021) reported that the generic antioxidant effects of SODs are beneficial in all conditions tested, from ocular and cardiovascular diseases to neurodegenerative disorders and metabolic diseases, including diabetes and its complications and obesity. However, according to the authors, clinical evidence of its effectiveness is still limited and, consequently, its effectiveness still needs to be fully demonstrated (Molecules. 2021 April; 26(7): 1844. Rosa et al.).

The use of SOD as a medicine has been seen as advantageous by the scientific community in terms of the quantity and duration of the pharmacological effect when compared to other antioxidants. However, pharmacological treatment with exogenous administration of SOD is not yet a well-established clinical practice. In this case, dietary supplementation is usually sought (Molecules. 2021 April; 26(7): 1844. Rosa et al.). Another fact is that the effectiveness depends on the source of the SOD. Some comparative studies indicated that human and bovine SOD conferred greater pharmacological activity than the rat enzyme (Biochem. Pharmacol. 1984;33:2755-2760. Baret et al.). Curiously, other studies have stated that treating human diseases with human SOD may not produce beneficial effects. On the other hand, bovine SOD, known as orgoteine, was generally preferred. However, the limitation of treatments due to their intramuscular administration, and frequency of administration (2 to 3 times a week) [Clin. Pharmacokinet. 1995;28:17-25. Jadot et al.] as well as their possible toxicity, caused by the presence of 20% of impurities (albumin and chymotrypsin are the primary contaminants), in pharmaceutical preparation may result in immediate hypersensitivity reactions Allergol. Immunopathol. 2001;29:272-275. De Benito et al. and other side effects, including allergy [Nutrition. 2015;31:430-436. Romao S.]. For this reason, orgotein, marketed for the treatment of several inflammatory diseases, was withdrawn from European countries [Allergol. Immunopathol. 2001;29:272-275. De Benito et al.] due to allergic reactions and limited to veterinary use in the USA [Molecules. 2021 April; 26(7): 1844. Rosa et al.].

Orgoteine offers the advantage of having a high concentration of SOD (100 U/mg) and a low content of other antioxidants, such as CAT (10 U/mg) and GSH (1 U/mg), being one of the most used (Ethnopharmacol. 2004;94:67-75. Vouldoukis. Et al.; Food Chem. 2012;135:1298-1302. Carillon et al.). However, the oral bioavailability of this form of SOD is still very low, following the general pharmacokinetic principle of drugs, and this is due to its high molecular weight, which affects cellular uptake (Funct. Foods. 2020;68:103917. Stephenie et al.), and the low pH and high proteolytic activity in the digestive tract (Phytother. Res. 2004;18:957-962. Vouldoukis et al.). As natural SOD is an exogenous protein, we can hypothesize that it induce the formation of can antibodies (anti-ADA drug antibodies). However, considerable experience with the infusion of proteins as medicines for therapeutic purposes has indicated that there is only a marginal reduction in their effect and no clinically demonstrated toxicity (Molecules. 2021 April; 26(7): 1844. Rosa et al.).

The use of SOD mimetics (synthetic form of SOD) as well as new delivery systems to protect SOD by increasing its bioavailability are under investigation in the scientific community (Int. J. Biol. Macromol. 2020;168:846-865. Rosa et al.). On the one hand, SOD mimetics are intended to overcome the limits of natural SOD enzymes. They present better pharmacokinetic properties and some pharmacodynamic differences, with negligible potential antigenicity. SOD mimetics have low molecular weight, greater stability, and longer circulation half-life, ensuring a better pharmacokinetic profile. Furthermore, they present a different dose-response curve; natural SOD exhibits a bell-shaped dose-dependent curve, whereas most SOD mimetics have a dose-proportional response (Chemistry. 2018;24:5032-5041. Bonetta R.). Studies state that the mechanism of action of SOD goes far beyond the ·O2— scavenging activity alone, representing a promising potential for future therapies (Molecules. 2021 April; 26(7): 1844. Rosa et al.).

SOD exists in three forms, SOD 1 (cytoplasmic), SOD 2 (mitochondrial) and SOD 3 (specific in muscle tissue), that share the presence of binding sites for several transcription factors, such as NF-κB, the specificity protein (Sp)-1, CCAAT Enhancer Binding Proteins (C/EBP), and the activator proteins (AP)-1 and -2, which exert effects on the regulation of all three SOD genes (Free Radic. Biol. Med. 2009;47:344-356. Miao & Clair; Neurol. Res. Int. 2011;2011:458427. Milani et al.; Endocrinol. Diabetes. 2016;3:1-5. Houldsworth A.). The first evidence between nuclear factor erythroid 2-related factor 2 (Nrf2) concerning SOD1 was demonstrated in 2005 when a mutation in the SODG93A gene was associated with a reduction in Nrf2 mRNA (Brain. 2005;128 Pt 7:1686-1706. Kirby et al.).

Nrf2 translocates to the nucleus from the cytoplasm after binding with Kelch-like ECH-associated protein 1 (Keap1). Keap1 is a cysteine-rich protein that interacts with ROS and promotes nuclear translocation, ubiquitination, and degradation of Nrf2. The Keap1/Nrf2 pathway regulates the expression of many antioxidant genes in addition to SODs and can be considered the effector of the SOD mimetic mechanism of action. SOD mimetics alter the cysteine oxidation/S protein glutathionylation cycle in Keap1, thereby inducing Nrf2 activation and leading to SOD overexpression (Redox Biol. 2019;25:101139. Batinic-Haberle & Tome). The Keap1/Nrf2/HO-1 axis and its connection with SOD expression can be explained in the complementary function of SOD and HO-1; the first produces H2O2 and the second catalyzes the rate-limiting step of heme degradation into bilirubin (Ann. N. Y. Acad. Sci. 2008;1147:61-69. Johnson et al.), which is known to remove ROS, including OH, singlet oxygen, and O2 {Cell. Signal. 2014;26:512-520. Quaisiya et al.).

Piantadosi et al. demonstrated that Nrf2/HO-1 confers protection against doxorubicin-induced mitochondrial damage by upregulating antioxidant genes, including SOD2 itself (Circ. Res. 2008;103:1232-1240. Piantadosi et al.0. Considering the role of Keap1/Nrf2 in SOD expression, Nrf2 activators, or Keap1 inhibitors (Cell. Longev. 2019;2019:9372182. Robledinos-Anton et al.), should be included among SOD inducers, among them, peroxisome proliferator-activated receptor-gamma (PPAR)γ being a particularly promising role in the regulation of SOD expression.

### SUMMARY OF THE INVENTION

Embodiments of the invention provide modified metal-organic frameworks (MOF), materials and compositions comprising the modified metal-organic frameworks (MOF) and uses of the materials and compositions comprising modified MOFs. The modified MOFs may include a functionalizing constituent that provides enhanced functionality, such as transporting molecules across biological membranes. Embodiments of the modified MOF may comprise a magnesium-gallate (Mg-GA) network structure. The Mg-GA MOF may also comprise phosphate-functionalized polyethylene glycol, which comprises PEGylates (polyethylene glycol) with phosphate groups.

Accordingly, it is an objective of the invention to provide MOF and MOF materials or compositions.

It is a further objective of the invention to provide methods of using MOF materials or compositions.

It is yet another objective of the invention to provide modified MOF and modified MOF materials or compositions.

It is a still further objective of the invention to provide methods of using modified MOF materials or compositions.

It is a further objective of the invention to provide modified MOF and modified MOF materials or compositions using a magnesium-gallium framework.

It is a further objective of the invention to provide uses of modified MOF and modified MOF materials or compositions using a magnesium-gallium framework.

It is an objective of the invention to provide modified MOF and modified MOF materials or compositions comprising phosphate-functionalized polyethylene glycol.

It is an objective of the invention to provide uses of modified MOF and modified MOF materials or compositions comprising phosphate-functionalized polyethylene glycol.

It is yet another objective of the invention to provide modified MOF and modified MOF materials or compositions comprising Nicotinamide Adenine Dinucleotide (NAD)-loaded magnesium-gallate.

It is a further objective of the invention to provide methods of using MOF materials or compositions comprising Nicotinamide Adenine Dinucleotide (NAD)-loaded magnesium-gallate for aging-related conditions or diseases.

It is a further objective of the invention to provide methods of using MOF materials or compositions comprising Nicotinamide Adenine Dinucleotide (NAD)-loaded magnesium-gallate for aging-related conditions or diseases, such as metabolic syndrome.

It is a further objective of the invention to provide methods of using MOF materials or compositions comprising Nicotinamide Adenine Dinucleotide (NAD)-loaded magnesium-gallate for aging-related conditions or diseases, such as aging processes.

It is yet another objective of the invention to provide modified MOF and modified MOF materials or compositions comprising superoxide dismutase (SOD)-loaded magnesium-gallate.

It is a further objective of the invention to provide methods of using MOF materials or compositions comprising superoxide dismutase (SOD)-loaded magnesium-gallate for aging-related conditions or diseases.

It is a further objective of the invention to provide methods of using MOF materials or compositions comprising superoxide dismutase (SOD)-loaded magnesium-gallate for aging-related conditions or diseases, such as metabolic syndrome.

Other objectives and advantages of this invention will become apparent from following description the taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

### BRIEF DESCRIPTION OF THE FIGURES

FIG. 1 is an illustrative example of a modified MOF, according to some embodiments of the invention;

FIG. 2 illustrates an embodiment of the modified MOF illustrated in FIG. 1, shown with multiple functionalizing R₁ structures;

FIG. 3 illustrates an embodiment of the modified MOF illustrated in FIG. 1, shown with a phosphate-functionalized polyethylene glycol (mPEG-PO3);

FIG. 4 illustrates an embodiment of the modified MOF illustrated in FIG. 1, shown with a magnesium-gallate network and a phosphate-functionalized polyethylene glycol (mPEG-PO3);

FIG. 5 is an illustrative example of a modified MOF, according to some embodiments of the invention;

FIG. 6 illustrates an embodiment of the modified MOF illustrated in FIG. 5, shown with magnesium-gallate network and NAD;

FIG. 7A illustrates the binding of Mg-GA-NAD structure;

FIG. 7B illustrates the binding of Mg-GA-NAD-(mPEG-PO3) structure;

FIG. 8 illustrates an embodiment of the modified MOF illustrated in FIG. 5, shown with the Mg—Ga structure having multiple NADs attached to the Ga structures;

FIG. 9 illustrates an embodiment of the modified MOF illustrated in FIG. 5, shown attached with multiple NADs and functionalizing R1 structures;

FIG. 10 illustrates an embodiment of the modified MOF illustrated in FIG. 5, shown as a NAD loaded-Mg—Ga structure with phosphate-functionalized polyethylene glycol;

FIG. 11 is a schematic illustration of the modified MOF illustrated in FIG. 5, shown as a NAD loaded-Mg—Ga structure with phosphate-functionalized polyethylene glycol loaded to NAD;

FIG. 12 illustrates an embodiment of the modified MOF illustrated in FIG. 5, shown as a coated NAD loaded-Mg—Ga structure with phosphate-functionalized polyethylene glycol loaded to NAD;

FIG. 13 is an illustrative example of a modified MOF, according to some embodiments of the invention;

FIG. 14 illustrates an embodiment of the modified MOF illustrated in FIG. 13, shown with magnesium-gallate network and SOD;

FIG. 15 illustrates the binding of Mg-GA-SOD structure;

FIG. 16 illustrates an embodiment of the modified MOF illustrated in FIG. 13, shown with the Mg—Ga structure having multiple SODs attached to the Ga structures;

FIG. 17 illustrates an embodiment of the modified MOF illustrated in FIG. 13, shown attached with multiple SODs and functionalizing R1 structures;

FIG. 18 illustrates an embodiment of the modified MOF illustrated in FIG. 13, shown as a SOD loaded-Mg—Ga structure with phosphate-functionalized polyethylene glycol;

FIG. 19 is an illustrative example of a modified MOF, according to some embodiments of the invention, illustrated as a quercetin-loaded MG—Ga MOF;

FIG. 20 is an illustrative example of a modified MOF, according to some embodiments of the invention, illustrated as a Ursolic Acid-loaded MG—Ga MOF;

FIG. 21 is an illustrative example of a modified MOF, according to some embodiments of the invention, illustrated as a Alpha Lipoic Acid-loaded MG—Ga MOF;

FIG. 22 is an illustrative example of a modified MOF, according to some embodiments of the invention, illustrated as a pyrroloquinoline quinone (PQQ)-loaded MG—Ga MOF;

FIG. 23 is an illustrative example of a modified MOF, according to some embodiments of the invention, illustrated as a ubiquinol/CoQ10-loaded MG—Ga MOF;

FIG. 24 is an illustrative example of a modified MOF, according to some embodiments of the invention, illustrated as a Fisetin-loaded MG—Ga MOF;

FIG. 25 is an illustrative example of a modified MOF, according to some embodiments of the invention, illustrated as a glutathione-loaded MG—Ga MOF;

FIG. 26 is an illustrative example of a modified MOF, according to some embodiments of the invention, illustrated as a SOD1/SOD2 (80/20)-loaded MG-Ga MOF;

FIG. 27 is an illustrative example of a modified MOF, according to some embodiments of the invention, illustrated as a astaxanthin-loaded MG-Ga MOF;

FIG. 28 is an illustrative example of a modified MOF, according to some embodiments of the invention, illustrated as a Beta-caryophellene-loaded MG-Ga MOF;

FIG. 29 is an illustrative example of a modified MOF, according to some embodiments of the invention, illustrated as a berberine-loaded MG-Ga MOF;

FIG. 30 is an illustrative example of a modified MOF, according to some embodiments of the invention, illustrated as a testosterone-loaded MG-Ga MOF;

FIG. 31 is an illustrative example of a modified MOF, according to some embodiments of the invention, illustrated as a resveratrol-loaded MG-Ga MOF, see FIG. 33;

FIG. 32 is an illustrative example of a modified MOF, according to some embodiments of the invention, illustrated as a carnosine-loaded MG-Ga MOF;

FIG. 33 is a table (Table 1) providing data related to the PK analysis in brain and blood; and

FIG. 34 is a table (Table 2) providing data related to bioanalytical results in the brain.

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

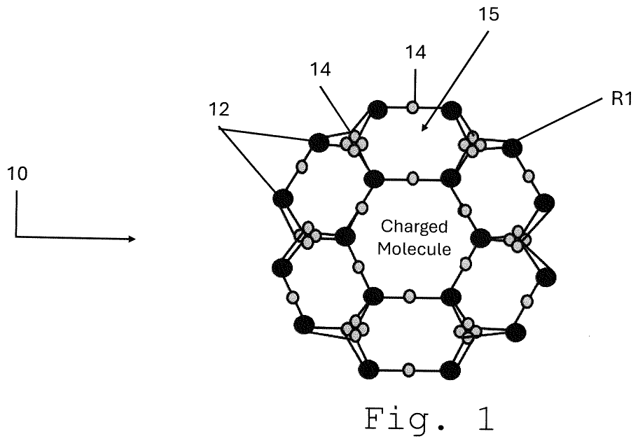

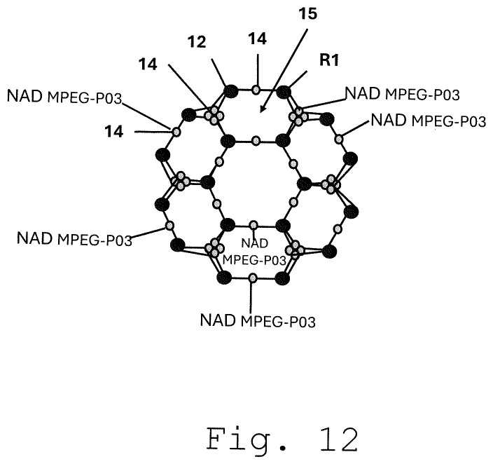

Referring to FIGS. 1-36, embodiments of modified metal-organic frameworks (MOF), MOF materials and compositions comprising the MOF, and uses of the materials and compositions comprising modified MOFs, are provided. In certain embodiments, the modified MOFs may comprise one, two, or three-dimensional coordination polymer having metal ions and ligands, together functioning as an organic structural unit.

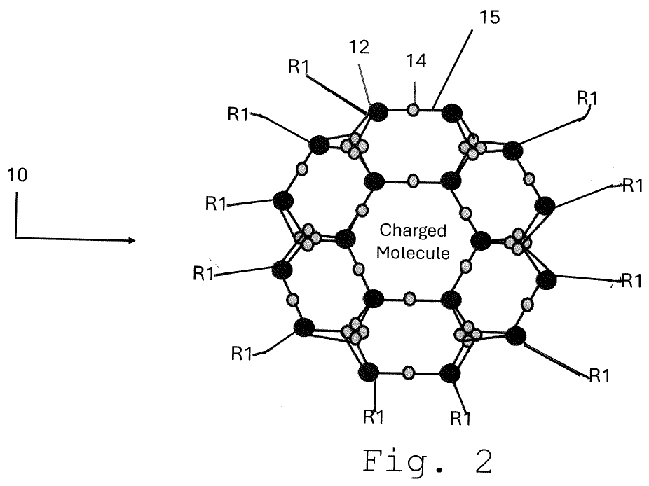

Referring to FIG. 1, an illustrative example of a modified MOF, referred to generally as modified MOF 10, is illustrated. The modified MOF 10 comprises a plurality of metal ions 12, also referred to as metal nodes and ligands 14. In certain embodiments, the ligands 14 may be organic ligands. The modified MOF 10 may be synthesized so the plurality of metal ions 12 and ligands 14 are arranged to form a structure having pores 15. In certain embodiments, the modified MOF 10 further comprises at least one functionalizing structure R₁. In certain embodiments, the R₁ structure(s) is a complete hybridization of the molecule forming the three-dimensional structure, which allows for the formation of micropores. This functional structure R₁ could form the skeleton of the molecule. This synthesis can be done by ionic bonds through hydrothermal reactions in specific reactors. In certain embodiments, the modified MOF 10 comprises a plurality of functionalizing structures R₁, this forming a “coating” around the structure, see FIG. 2. While illustrated having the R₁ structures attached to all of the metal ions 12 along the outer perimeter of the modified MOF 10, the modified MOF 10 may include all metal ions 12 bound to an R₁ structure, or less than all metal ions 12 bound to an R₁ structure, such as between 1% and 99%, including any value between 1% and 9%, any value between 10% and 19%, any value between 20% and 29%, any value between 30% and 39%, any value between 40% and 49%, any value between 50% and 59%, any value between 60% and 69%, any value between 70% and 79%, any value between 80% and 89%, and any value between 90% and 99%. Alternatively, the modified MOF 10 may comprise each of, or, one or more of the metal ions 12 attached to two or more R₁ structures.

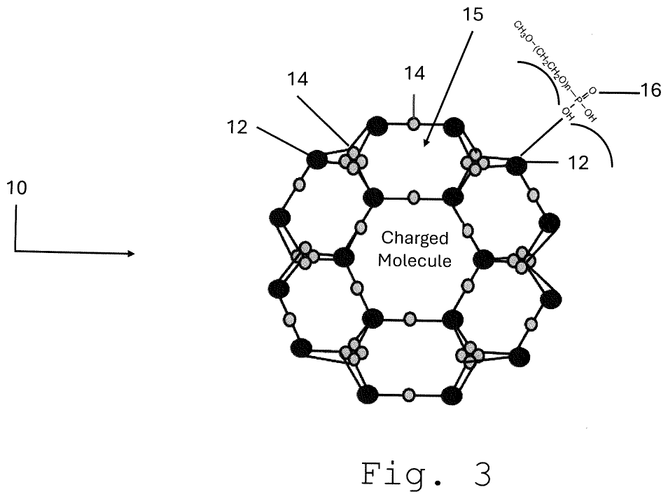

In certain embodiments, the functionalizing structure R₁ is PEGylates (polyethylene glycol), with the presence of phosphate groups. Referring to FIG. 3, an embodiment of the modified MOF 10 is shown with a phosphate-functionalized polyethylene glycol (mPEG-PO3) 16. Addition of the phosphate-functionalized polyethylene glycol 16 to MOF 10 is believed to 1) improve the conjugation of organic compounds, 2) load any organic compound that contains phosphate groups, because the phosphate ions bind to the PEGylate effectively, improving the ability of the MOF 10 to load any ingredient, providing better stability to the MOF, and 3) control of the release mechanism of the loaded product to the MOF (PEGylate attached/bonded with MOF makes it possible to control release while maintaining a more linear curve when it comes to pharmacokinetics). MOF 10 may therefore be used as a vehicle to transport molecules across biological membranes.

In certain embodiments, the modified MOF 10 may comprise a magnesium-gallate (Mg-GA) network structure; the magnesium-gallate (Mg-GA) network structure being a positively charged, one-dimensional, two-dimensional, or three-dimensional structure of Magnesium, an alkaline earth metal having symbol Mg and atomic number 12, and Gallate, a salt or ester of gallic acid. Gallic acid, also known as 3,4,5-trihydroxybenzoic acid, is a trihydroxybenzoic acid having the formula C₆H₂(OH)₃CO₂H, and classified as phenolic acid. Use of Mg-GA may offer several advantages. Gallic acid is known to have advantages over expensive organic ligands due to low cost, low toxicity, easy availability and natural abundance (J. Pharmacogn. Phytochem. 2016, 4, 35-49. Nayamai et al.). Studies have already proven that gallic acid can act as a successful alternative organic ligand in a new family of hybrid framework materials known as gallate or Mg-gallate-based MOFs (Dalton Trans. 2011, 40, 6401-6410. Saines et al.). This gallic acid is obtained from the production of biomass on an industrial scale at a much more affordable cost (J. Am. Chem. Soc. 2017, 139, 7733-7736. Li et al.). Furthermore, the entire synthetic process is environmentally friendly without the consumption of organic solvents (Angew. Chem. 2018, 130, 16252-16257. Bao et al.). At first, gallate-based MOFs were widely used in biomedical applications. Gallate-based MOFs gradually have been used in small scopes of chemical applications, such as light hydrocarbon separations, due to their excellent performance (Crystals 2020, 10(11), 1006; Ismail et al.). Thus, gallic acid has the potential to be an excellent organic ligand for gallate-based MOFs due to its environmentally and d economically friendly nature.

One of the significant features of gallate-based MOFs is that they can contain divalent and trivalent cations while maintaining charge balance (Solid State Sci. 2006, 8, 1121-1125. Feller & Cheetham). Furthermore, gallate-based MOFs have been proven to have high stability against water and oxygen, as well as recyclability as they can be applied in repetitive adsorption-desorption cycles that are important for real-world applications (Chem. A Eur. J. 2019, 25, 15516-15524. Wang et al.). Due to the various biological effects of gallic acid, gallate-based MOFs have been applied in biomedical applications, such as antioxidant carriers and anticancer agents.

Other features of using gallate-based MOFs includes low cost for large-scale production, versatility in biological environments (high bioavailability when linked to magnesium), good synergy with other organic-to-be-loaded ingredients to control oxidative and inflammatory processes caused by age, adaptogenic properties such as distinct action on different types of tissue according to pH (which allows it to be used in different pathologies), resists the pH of the digestive system without compromising the ingredient loaded, and easy to control the release of the ingredients loaded in your pores into the target tissues, make Mg-GA a promising option, both in increasing the bioavailability of drugs/nutraceuticals and its therapeutic action.

Similarly, gallate can be complexed with magnesium forming a hybrid structure, where micropores are created, facilitating the covalent bond mentioned above, being able to protect and transport the loaded ingredient to biological environments without compromising bioavailability (Crystals 2020, 10(11), 1006. Ismail. Et al.). Gallate as MOFs can protect the molecule from dissociating in very acidic pH, as occurs in the digestive process in the stomach, protecting the molecule from the degradative action of hydrochloric acid (Crystals 2020, 10(11), 1006; Ismail et al.). In more alkaline environments, such as of inside cells, the possibility degradation is greater, allowing the molecule to disintegrate and deliver its loaded ingredients intact. Therefore, to form magnesium gallate as MOF, magnesium chloride is an efficient form of magnesium to be used in a hydrothermal reaction to be complexed with gallate to form magnesium-gallate in the carboxyl group mentioned above.

Magnesium has several biological functions. Magnesium is crucial in the processes of protein translation, playing a vital role in ribosome function and protein synthesis. Magnesium acts as an enzymatic cofactor of ATP synthase, being crucial in both ATP synthesis and ATP hydrolysis. Magnesium acts as an enzymatic cofactor in the glycolytic pathway in anaerobic metabolism. Magnesium acts as an enzymatic cofactor in several enzymes in the central nervous system that act in the synthesis of serotonin, catecholamines and GABA, helping the metabolism of the brain in general. Magnesium has a crucial action in DNA synthesis, mainly on DNA polymerase, which has a catalytic action in the synthesis and stabilization of DNA.

Gallic acid has several biological functions. Gallic acid is a potent activator of nuclear factor erythroid 2-related factor 2 (Nrf2), a crucial transcription factor that regulates the expression of antioxidant enzymes. Gallic acid inhibits proinflammatory cytokines by preventing NF-κB activation and lowering the inflammatory response. Gallic acid activates numerous protein kinases, including phosphoinositide 3 kinase (PI3K), protein kinase B (Akt), mitogen-activated protein kinase (MAPK) and adenosine monophosphate-activated protein kinase (AMPK). By suppressing the MAPK pathway, gallic acid reduces NF-κB activation, which in turn lowers the expression of pro-inflammatory cytokines and other mediators, thus decreasing inflammation and oxidative stress.

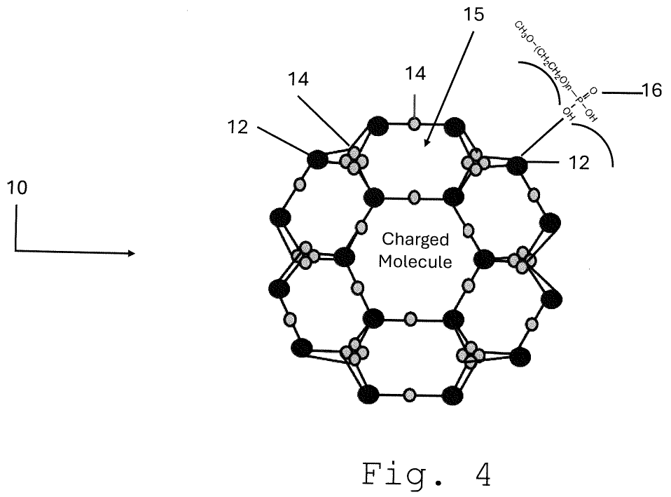

In certain embodiments, the modified MOF 10 may comprise phosphate-functionalized polyethylene glycol (mPEG-PO3)/Mg-GA structure. As illustrated in FIG. 4, the modified MOF 10 is shown with phosphate-functionalized polyethylene glycol (mPEG-PO3) 16 being attached to metal ion 12′, representing Mg and the organic ligand 14′, representing gallate. The modified MOF 10 may be a nano-molecule of up to 500 nm, such as 50-500 nm.

In certain embodiments of the invention, using a post-synthetic modification of phosphate-functionalized polyethylene glycol (mPEG-PO3) nanoMOFs/Mg-GA 10 combined with freeze-drying could lead to the formation of re-dispersible solid materials. This approach can serve a efficient method for storing single or drug/nutraceutical-loaded nanoMOFs. The PEGylated nanoMOFs can exhibit stable hydrodynamic diameters, improved colloidal stability, and delayed drug/nutraceutical release kinetics compared to their pristine nanoMOFs. Freeze-dried and PEGylated nanoMOFs can be completely redispersed in water, avoiding common aggregation issues that would limit the use of MOFs in the biomedical field to wet form—a critical limitation for their translation to clinical use, as these materials can now be stored as dry samples. mPEG-PO3, through its phosphate group, is believed to anchor itself to the metallic structure of the MOF on its surface, keeping most of the PEGylate outside the MOF pores attached to the metallic site forming the coating around the MOF structure.

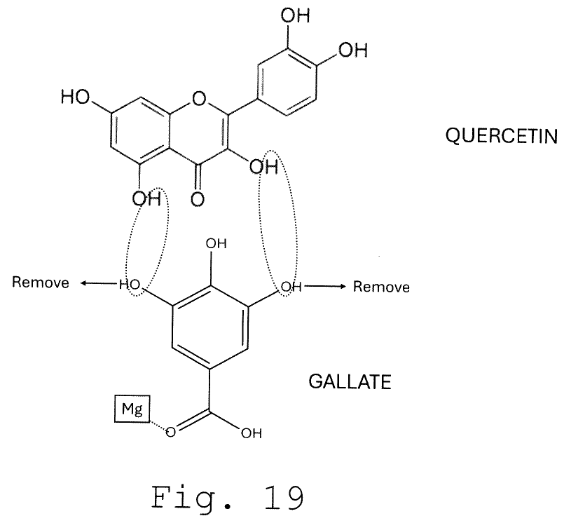

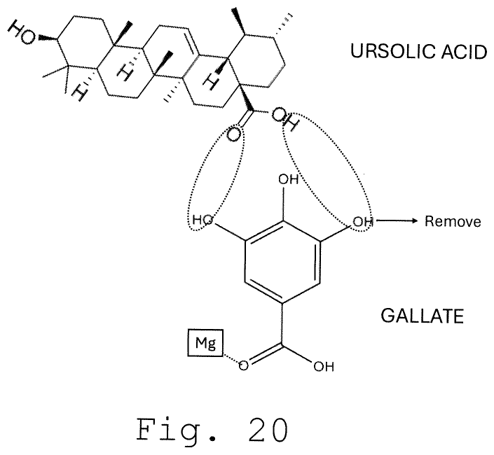

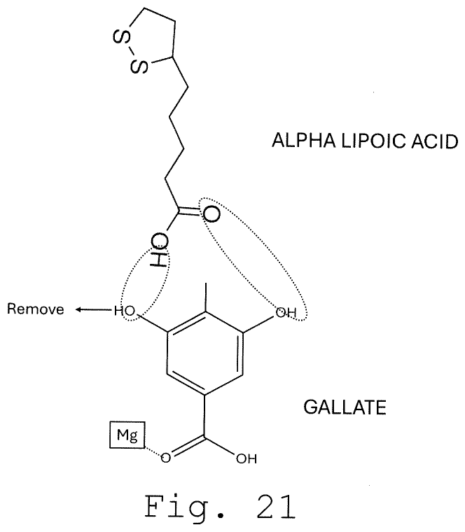

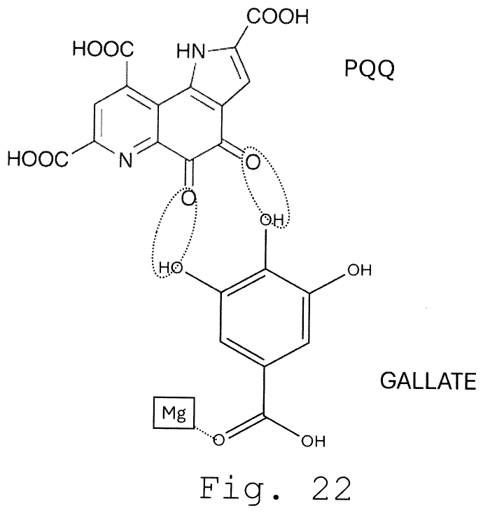

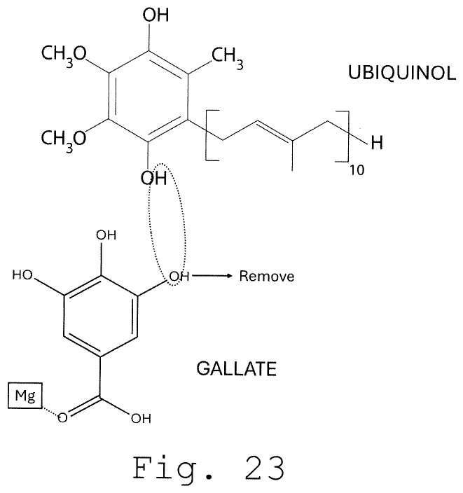

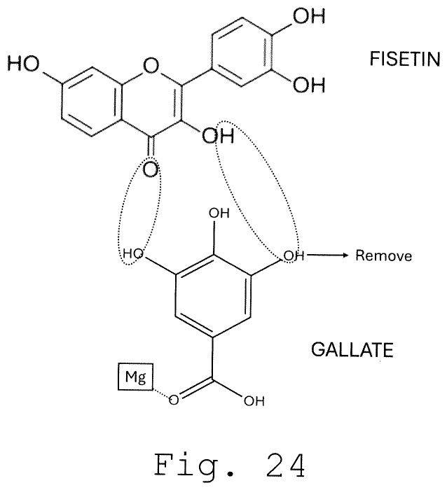

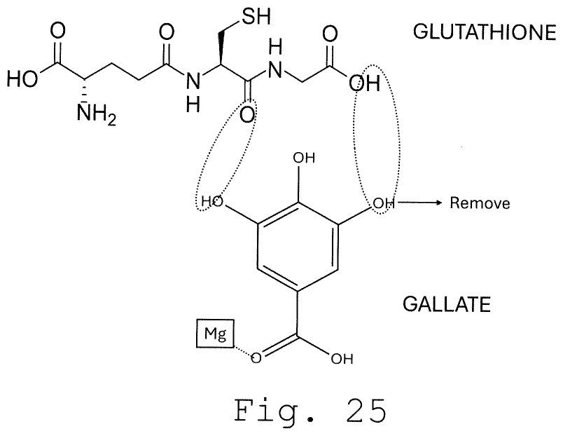

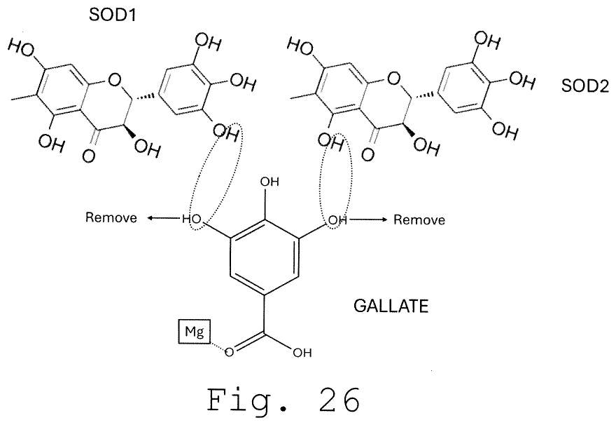

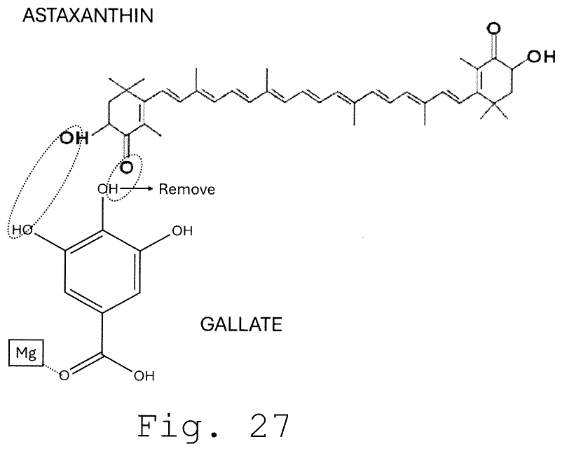

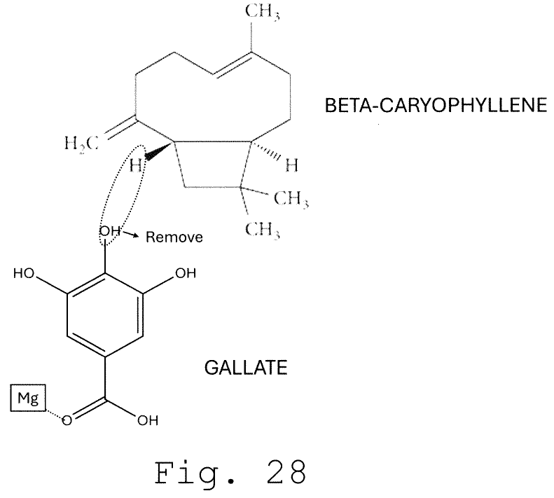

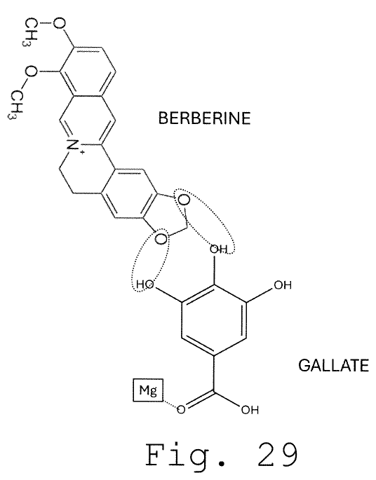

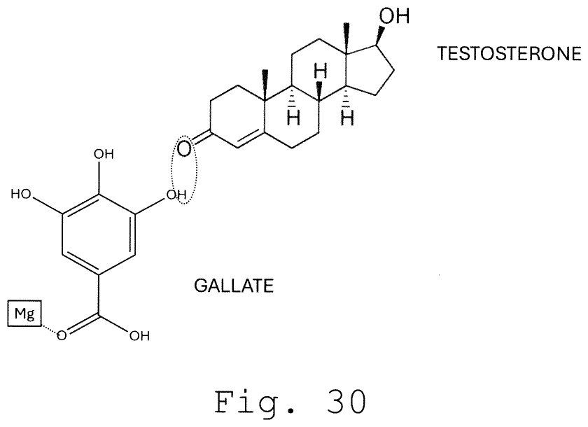

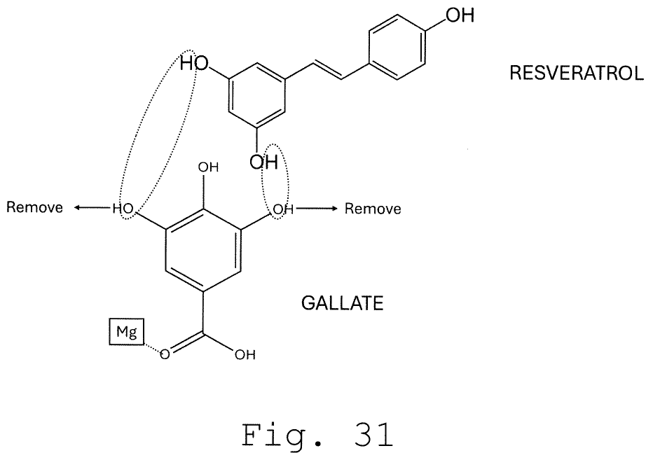

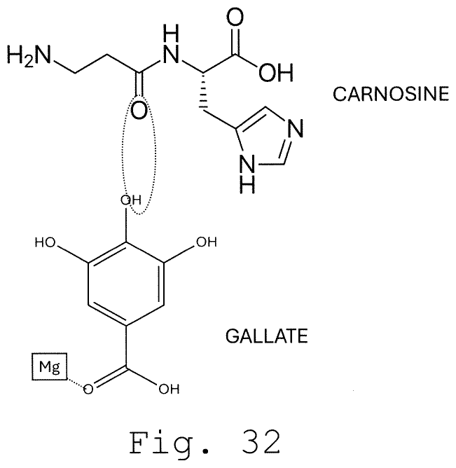

In certain embodiments, the phosphate-functionalized polyethylene glycol (mPEG-PO3) nanoMOFs/Mg-GA could be constructed to accommodate and distribute an organic molecule, such as but not limited to, coenzymes, enzymes, flavonoids, carotenoids, hormones, phytochemicals etc. Illustrated examples of such organic molecules loaded to the MOF may include quercetin-loaded MG-Ga MOF, see FIG. 19; Ursolic Acid-loaded MG-Ga MOF, see FIG. 20; Alpha Lipoic Acid-loaded MG-Ga MOF, see FIG. 21, pyrroloquinoline quinone (PQQ)-loaded MG-Ga MOF, see FIG. 22; ubiquinol/CoQ10-loaded MG-Ga MOF, see FIG. 23; Fisetin-loaded MG-Ga MOF, see FIG. 24; glutathione-loaded MG-Ga MOF, see FIG. 25; SOD1/S) D2 (80/20)-loaded MG-Ga MOF, see FIG. 26; astaxanthin-loaded MG-Ga MOF, see FIG. 27; Beta-caryophellene-loaded MG-Ga MOF, see FIG. 28; berberine-loaded MG-Ga MOF, see FIG. 29; testosterone-loaded MG-Ga MOF, see FIG. 30; resveratrol-loaded MG-Ga MOF, see FIG. 31; carnosine-loaded MG-Ga MOF, see FIG. 32. The modified MOF 10 with phosphate-functionalized polyethylene glycol (mPEG-PO3)/Mg-GA structure, being a positively charged material, is believed to provide a higher absorption rate when interacting with negatively charged cell membranes. As such, the modified MOF 10 with phosphate-functionalized polyethylene glycol (mPEG-PO3)/Mg-GA structure could fit perfectly into endocytosis mechanisms and distribute any drug/nutraceutical into a biological environment without the limitations of other methods. Thus, the targeted release mechanisms of drugs/nutraceuticals loaded into the modified MOF 10 with phosphate-functionalized polyethylene glycol (mPEG-PO3)/Mg-GA, such as with a nanoparticle between 50 to 500 nm, is believed to provide benefits relating to both pharmacokinetics and pharmacodynamics of any ingredients loaded in this structure.

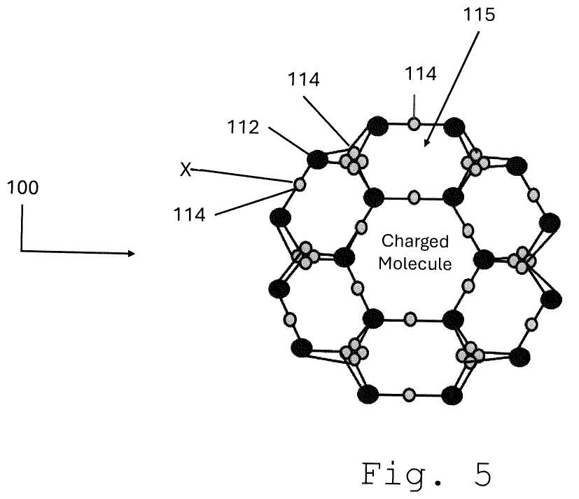

Referring to FIGS. 5-12, embodiments of the modified metal-organic frameworks (MOF) comprising a magnesium-gallate (Mg-GA) network structure loaded with Nicotinamide Adenine Dinucleotide (NAD) are illustrated. Biologically, NAD is involved in the transfer of electrons in the respiratory chain. When it receives hydrogen, it becomes reduced (NADH) initiating the transport of electrons between hydrogen and oxygen, thus allowing the synthesis of ATP. NAD is involved in the preservation of genome stability, in the response to cellular DNA damage, and in other pathways that regulate nucleic acid metabolism, such as gene expression and cell proliferation pathways. NAD is also involved as a substrate for ADP-ribosyltransferases, sirtuins, and potentially DNA ligases, all of which regulate various aspects of DNA integrity, damage repair, and gene expression. As NAD+boosts the activity of Sirtuin1 and other sirtuins, intracellular levels of NAD+ play a key role in the homeostatic control of mitochondrial function by the metabolic status of the cell.

Referring to FIG. 5, an embodiment of a modified MOF, referred to generally as modified MOF 100, is provided. The modified MOF 100 comprises a plurality of metal ions 112, also referred to as metal nodes, and ligands 114. The modified MOF 100 may be synthesized so the plurality of metal ions 112 and ligands 114 are arranged to form a structure having pores 115. The modified MOF 100 further comprises a loading molecule X attached to the ligand 114. The modified MOF 100 may be a nano-molecule of up to 500 nm, such as 50-500 nm.

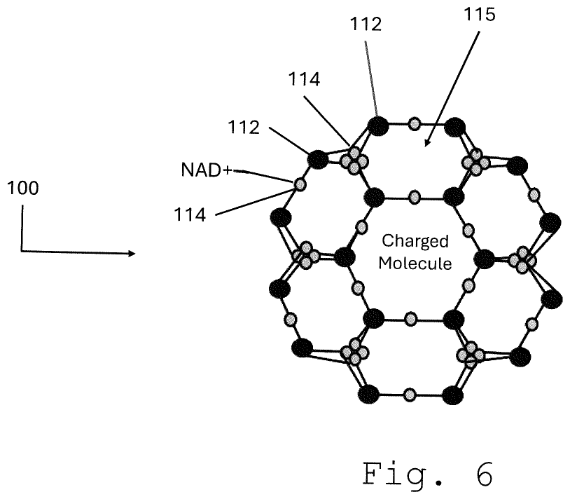

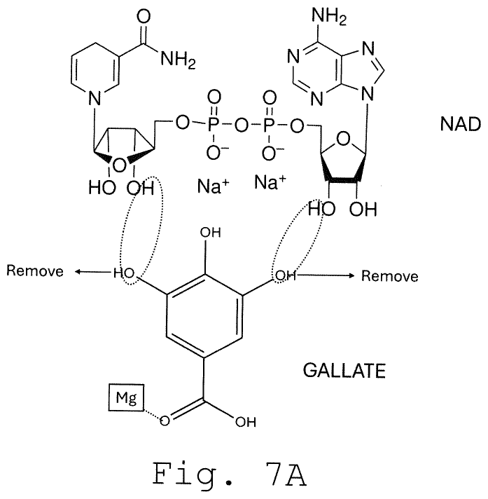

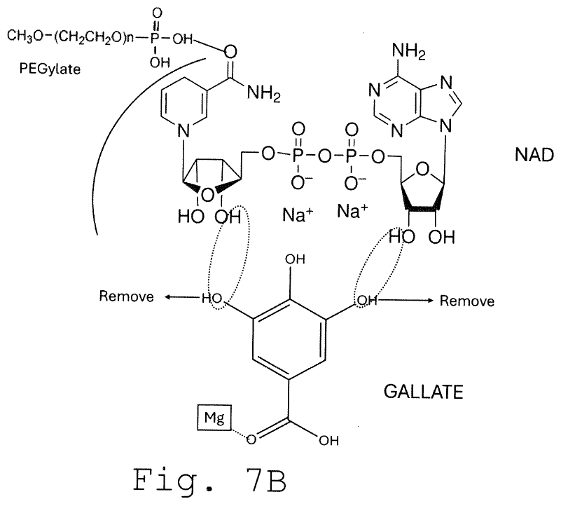

Referring to FIG. 6, in certain embodiments, the modified MOF 100 may comprise a magnesium-gallate (Mg-GA) network structure loaded with NAD; the magnesium-gallate (Mg-GA) network structure being a positively charged, one-dimensional, two-dimensional, or three-dimensional structure of Magnesium, an alkaline earth metal having symbol Mg and atomic number 12, and Gallate, a salt or ester of gallic acid. Gallic acid, also known as 3,4,5-trihydroxybenzoic acid, is a trihydroxybenzoic acid having the formula C₆H₂(OH)₃CO₂H, and is classified as phenolic acid. Accordingly, in FIG. 6, metal ions 112 would represent magnesium and ligands 114 would represent gallate. The loading molecule X is Nicotinamide Adenine Dinucleotide (NAD). As illustrated in FIG. 7A, NAD is shown chemically bound to the Mg—Ga structure, where the Oxygen (O) of the phenolic ring structure of gallate binds with the (OH) associated with ribose structures of NAD.

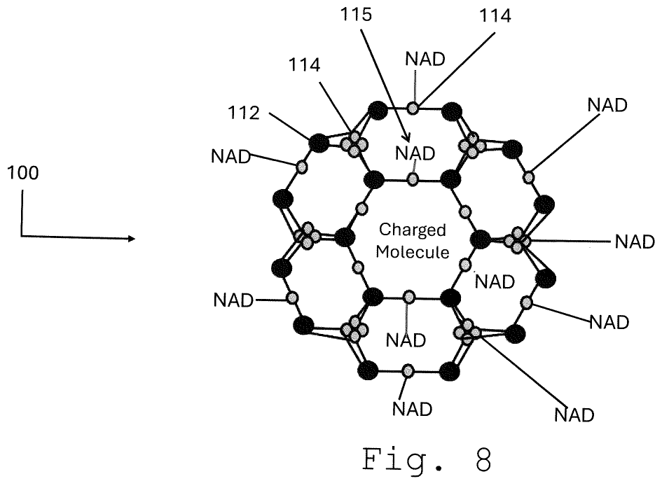

In certain embodiments, the modified MOF 100 comprises a plurality of loading molecules X, illustrated as multiple NAD molecules, thus forming a “coating” around the structure, see FIG. 8. FIG. 8 represents the Mg—Ga structure MOF 100 having multiple, but not all (100%), NADs attached to the modified Ga structures. The modified MOF 100 may be synthesized to have 100% binding of NAD-Mg or any value less than 100%, such as between 1% and 99%, including any value between 1% and 9%, any value between 10% and 19%, any value between 20% and 29%, any value between 30% and 39%, any value between 40% and 49%, any value between 50% and 59%, any value between 60% and 69%, any value between 70% and 79%, any value between 80% and 89%, and any value between 90% and 99%.

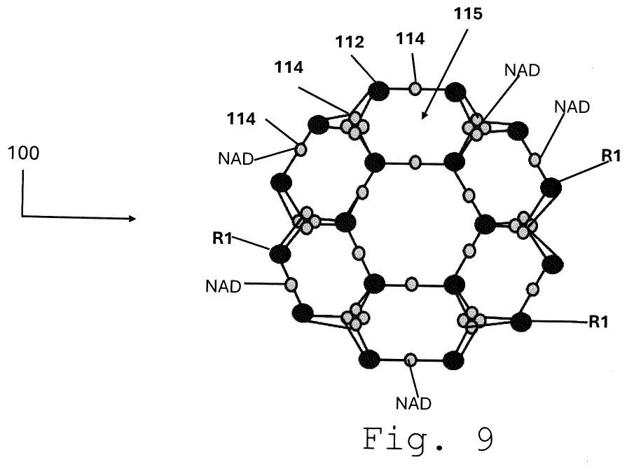

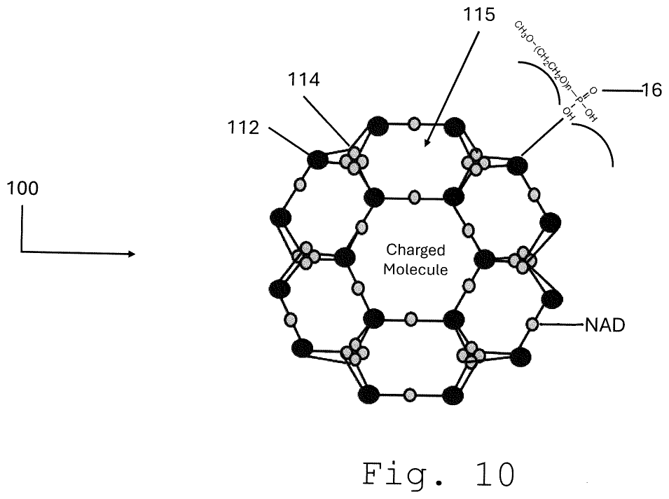

In certain embodiments, the modified MOF 100 comprises one or more functionalizing structure(s) R₁ attached to the metal ion 112, see FIG. 9. FIG. 10 represents the modified MOF 100 with NAD attached, where the R₁ structure(s) are illustrated as PEGylates (polyethylene glycol), with the presence of phosphate groups, thus forming a phosphate-functionalized polyethylene glycol (mPEG-PO3)/Mg-GA/NAD structure.

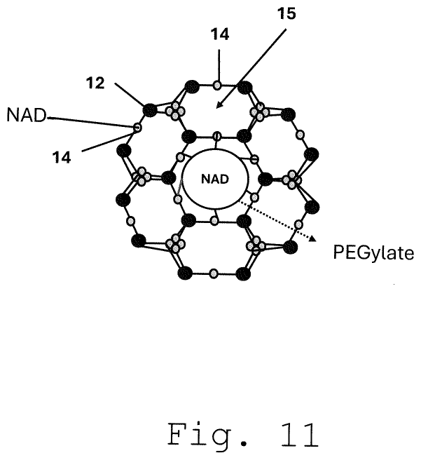

In certain embodiments, the modified MOF 100 comprises one or more functionalizing structure(s) R₁ attached or loaded to NAD. FIG. 11 represents the modified MOF 100 where the R₁ structure(s), illustrated as PEGylates (polyethylene glycol), with the presence of phosphate groups, thus forming a phosphate-functionalized polyethylene glycol (mPEG-PO3)/Mg-GA/NAD structure, is/are attached (loaded) to the NAD. As illustrated in FIG. 7B, the NAD-(mPEG-PO3) structure is shown chemically bound to the Mg—Ga structure, where the Oxygen (O) of the phenolic ring structure of gallate binds with the (OH) associated with ribose structures of NAD.

In certain embodiments, the modified MOF 100 comprises a plurality of NAD-(mPEG-PO3), thus forming a “coating” around the structure, see FIG. 8. FIG. 8 represents the modified MOF 10 having multiple, but not all (100%), NAD-(mPEG-PO3)s attached to the modified Ga structures. The modified MOF 10 may be synthesized to have 100% binding of NAD-mPEG-PO3s-Mg or any value less than 100%, such as between 1% and 99%, including any value between 1% and 9%, any value between 10% and 19%, any value between 20% and 29%, any value between 30% and 39%, any value between 40% and 49%, any value between 50% and 59%, any value between 60% and 69%, any value between 70% and 79%, any value between 80% and 89%, and any value between 90% and 99%.

Using magnesium-gallate loaded with NAD or NAD-(mPEG-PO3) is believed to allow the modified MOF 10 to pass through the gastrointestinal tract intact and also cross biological barriers until reaching the target tissue, even concerning the brain and its protection by the blood-brain barrier. With the versatility in loading molecules within its pores, loading NAD/NAD-(mPEG-PO3) into the magnesium-gallate modified MOF 10 may provide a strategy that increases the bioavailability of NAD and enhances both the effects of gallate on molecular mechanisms and the efficiency from NAD itself. Finding a way to increase the bioavailability of oral NAD+ administration would be a promising strategy from a nutritional point of view to increase NAD levels in certain circumstances, such as aging, neurodegenerative diseases, heart disease and metabolic syndrome. Synthesizing NAD/NAD-mPEG-PO3 loaded into magnesium gallate in accordance with the embodiments of the invention could be a mechanism to achieve this since this molecule would cross not only the brush border of enterocytes but also the blood-brain barrier, increasing NAD levels in the central nervous system.

To treat any diseases in metabolic syndrome as well as aging control, it may be necessary to reduce inflammatory processes (NF-κβ), reduce mitochondrial ROS production, and promote mitochondrial biogenesis through the PGC-1a, AMPK and SIRT1. Use of the NAD-loaded magnesium gallate in accordance with the embodiments of the invention may promote an increase in the expression of these proteins, and control electron transport in the respiratory chain in mitochondria.

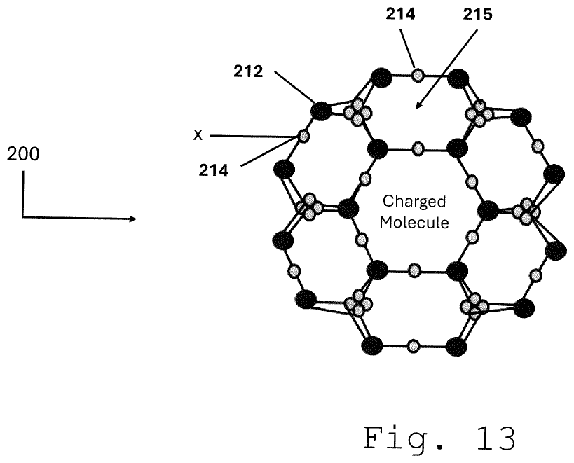

Referring to FIGS. 13-20, embodiments of the modified metal-organic frameworks (MOF) comprising a magnesium-gallate (Mg-GA) network structure loaded with superoxide dismutase (SOD)) are illustrated. Biologically, SOD is a key enzyme for the removal of superoxide, the most dangerous form of reactive oxygen species (ROS) produced cells through cellular respiration or by attacking pathogens on the cell membrane. SOD converts superoxide into hydrogen peroxide decreasing the production of superoxide in cells.

Referring to FIG. 13, an embodiment of a modified MOF, referred to generally as modified MOF 200, is provided. The modified MOF 200 comprises a plurality of metal ions 212, also referred to as metal nodes, and ligands 214. The modified MOF 200 may be synthesized so the plurality of metal ions 212 and ligands 214 are arranged to form a structure having pores 215. The modified MOF 200 further comprises a loading molecule X attached to the ligand. The modified MOF 200 may be a nano-molecule of up to 500 nm, such as 50-500 nm.

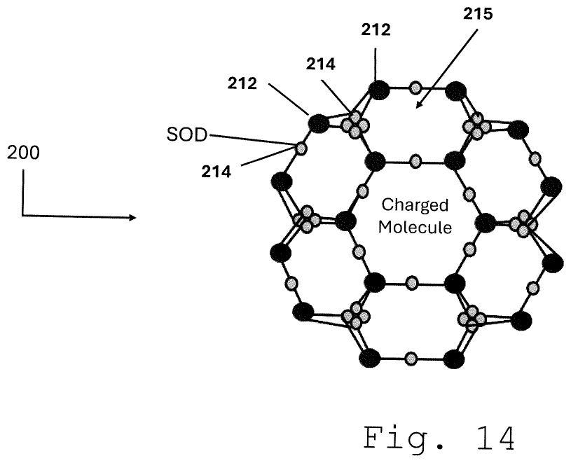

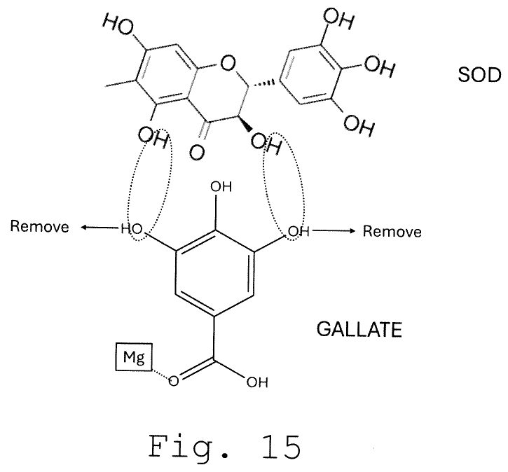

Referring to FIG. 14, in certain embodiments, the modified MOF 200 may comprise a magnesium-gallate (Mg-GA) network structure loaded with SOD; the magnesium-gallate (Mg-GA) network structure being a positively charged, one-dimensional, two-dimensional, or three-dimensional structure of Magnesium, an alkaline earth metal having symbol Mg and atomic number 12, and Gallate, a salt or ester of gallic acid. Accordingly, in FIG. 14, metal ions 212 represent magnesium and ligands 214 represent gallate (gallic acid). The loading molecule X is illustrated as SOD. As illustrated in FIG. 15, the SOD is shown chemically bound to the Mg—Ga structure, where the (OH) of the gallate structure binds with a double bonded Oxygen associated with SOD structure.

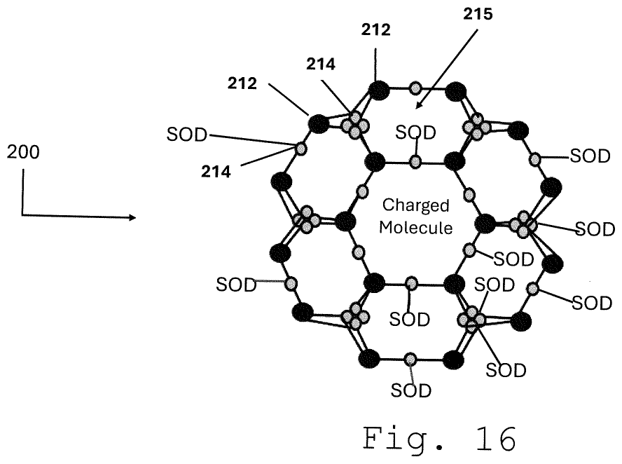

In certain embodiments, the modified MOF 200 comprises a plurality of loading molecules X, illustrated as multiple SOD molecules, thus forming a “coating” around the structure, see FIG. 16. FIG. 16 represents the Mg—Ga structure MOF 200 having multiple, but not all (100%), SODs attached to the modified Ga structures. The modified MOF 200 may be synthesized to have 100% binding of SOD-Ga or any value less than 100%, such as between 1% and 99%, including any value between 1% and 9%, any value between 10% and 19%, any value between 20% and 29%, any value between 30% and 39%, any value between 40% and 49%, any value between 50% and 59%, any value between 60% and 69%, any value between 70% and 79%, any value between 80% and 89%, and any value between 90% and 99%.

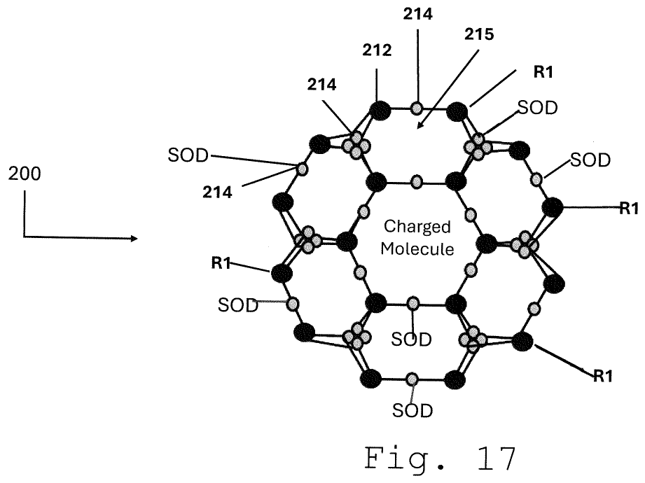

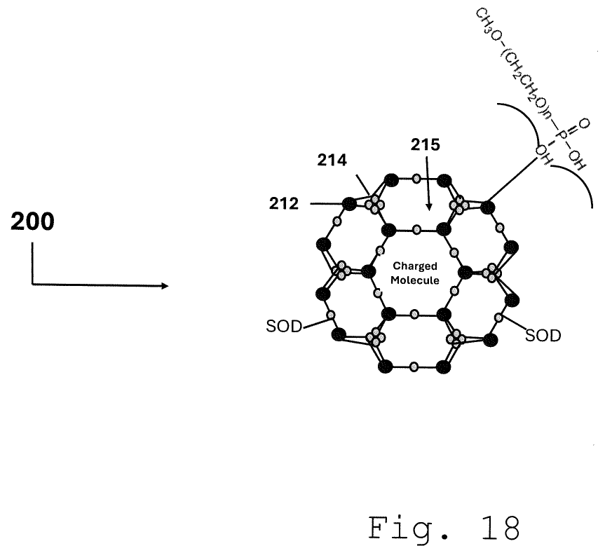

In certain embodiments, the modified MOF 200 comprises one or more functionalizing structure(s) R₁ attached to the metal ion 212, see FIG. 17. FIG. 18 represents the modified MOF 200 with SOD attached, where the R₁ structure(s) is/are illustrated as PEGylates (polyethylene glycol), with the presence of phosphate groups, thus forming a phosphate-functionalized polyethylene glycol (mPEG-PO3)/Mg-GA/SOD structure.

In certain embodiments, the modified MOF 200 comprises a plurality of SOD-(mPEG-PO3), thus forming a “coating” around the structure, see FIG. 8. FIG. 8 represents the modified MOF 200 having multiple, but not all (100%), SOD-(mPEG-PO3)s attached to the modified Mg—Ga structures. The modified MOF 200 Mg—Ga structure MOF 200 may be synthesized to have 100% binding of SOD-mPEG-PO3s-Mg or any value less than 100%, such as between 1% and 99%, including any value between 1% and 9%, any value between 10% and 19%, any value between 20% and 29%, any value between 30% and 39%, any value between 40% and 49%, any value between 50% and 59%, any value between 60% and 69%, any value between 70% and 79%, any value between 80% and 89%, and any value between 90% and 99%.

One of the defects in Metabolic syndrome and its associated diseases is excess cellular oxidative stress (reactive oxygen and nitrogen species, ROS/RNS) and oxidative damage to mitochondrial components, resulting in reduced efficiency of the electron transport chain developing a high inflammatory response. The use of flavonoids can attenuate this inflammatory response by modulating a specific protein called nuclear factor erythroid 2-related factor 2 (Nrf2). Gallic acid, for example, can restore oxidative and inflammatory states to normal levels through Nrf2. In response, cells control nuclear factor kappa-beta (NF-kB), thus reducing the inflammatory cascade of cytokines signaled by NF-kB itself. On the other hand, gallic acid, by positively signaling Nrf2, increases the expression of superoxide dismutase (SOD), improving the control of reactive oxygen species (ROS) and reactive nitrogen species (RNS). As SOD and Nrf2 have an interaction in metabolism seeking to maintain homeostasis, the presence of gallic acid and SOD in the same product would be promising from a metabolic point of view.

With the versatility in loading molecules within its pores, loading SOD into the magnesium-gallate MOF may not only increase the bioavailability of SOD but also enhance both the effects of gallate on molecular mechanisms and the efficiency from SOD itself. Since nutraceutical-based molecule that has the scientifically proven capacity regarding the pharmacokinetics of ingredients to treat metabolic syndrome in human metabolism currently exists, using an SOD-loaded magnesium gallate MOF may provide a promising strategy for this purpose, both in increasing bioavailability and in synergistic action on receptors and enzymes of human metabolism.

In certain embodiments, the modified MOF 200 may be used in methods of administering SOD to individuals in need, such as individuals suffering from metabolic syndrome diseases, such as Type 2 diabetes, heart disease, including heart failure, high blood pressure and cardiovascular inflammation, liver dysfunction, including hepatic steatosis and liver cirrhosis, and chronic fatigue, or neurological diseases, such as Parkinson's disease, or Amyotrophic lateral sclerosis.

Methods of synthesizing the modified MOF

Preparation of phosphate-functionalized polyethylene glycol MG-GA (FIGS. 1-4):

Step 1: Preparation of the Bivalent Cations (Mg, Zn, Ca).

Raw Materials
1. Magnesium chloride (anhydride) CAS #7786-30-3, lot #S8314333408 obtained from Sigma-Aldrich.
2. Gallic acid monohydrate, CAS #5996-86-8, lot #1003710701 from Sigma-Aldrich.
3. Distilled water obtained commercially.
4. Potassium Hydroxide (Axios Pharma)

A three-necked round-bottom flask equipped with a stirrer, thermometer, and condenser was charged with 250 ml of distilled water. The stirring started, and 11.40 g of gallic acid monohydrate (60.1 mm) was added. Stirring continued for 10 minutes.

Magnesium chloride 3 g (31.5 mmoles) was slowly added. Approximately 95% of the solids dissolved forming a beige solution. The pH of this solution was 3.8-4.0, and then adjusted to pH 8.0 at 25° C. with 10 M Potassium Hydroxide. The solution turned brownish in color and was clear. All the solids dissolved at this point, and the stirring continued while heating increased to 102° C., when refluxing started. The temperature was maintained at 102° C. for 24 hours. This solution was cooled and transferred into centrifuge tubes. It was centrifuged for thirty minutes at 4704 G Force (8000 rpm). Dupont Sorval CLC-28.

The top layer was decanted, and the tubes filled with the solids were washed with water (2×15 ml) and three times with ethanol. The solids were transferred to a China dish and dried at 75° C. for 6 hours. Yield 4.9 gms (˜78%).

Fourier-transform infrared (FTIR) Analysis (Perkin-Elmer Spectrum Two) confirmed the absence of Gallic Acid.

Step 2: A 500 mL three-neck round-bottom flask equipped with a stirrer, thermometer, and condenser was charged with 250 mL of distilled water. Gallic Acid 17.1 g [mmol] was slowly added and stirred for 10 minutes. 4.5 g magnesium chloride was added to this solution. This unique methodology was developed because of the current method's large volumes of water required. Mg/Ga was produced by converting gallic acid to its acid chloride (galloyl chloride) by treating it with Mg without solvents. After 3 hours of reflux, excess thionyl chloride evaporated, the product was washed with hexane, and finally with methanol.

The pH was adjusted to 8.0 with 10 M Potassium Hydroxide. Heating started and left overnight at 102-103° C. The next day it was cooled, centrifuged (Dupont Sorval CLC-28) at 8000 rpm. The top layer was decanted, and the precipitate was washed with 25 mL×2 water and 2×30 mL alcohol. The product was dried at 75° C. for three hours. Yield 9.0 g.

Step 3: Framework preparation of zinc/gallic acid.

To illustrate the framework, 8.71 g of Zinc chloride was charged into a 500 mL 3-neck flask. 250 ml of distilled water was added, followed by 11.4 g gallic acid monohydrate (0.12 M). Stirring continued for 10 minutes. The solution was observed to be slightly cloudy. The pH was adjusted to pH 10 using 10 M Potassium Hydroxide (˜40 mL). Initially, a precipitate formed but dissolved upon heating. The mixture was left overnight at 97-98° C., cooled, and filtered through a Buchner funnel. It was washed with water (3×50 mL) and Ethanol (2×50 ml), and the solids were dried under vacuum. Yield 24 g (80% of the product).

Pegylation of MG/G

Method 1: PEG Solution in Acetonitrile
1. 1 g of Mg/G was dispersed in 50 ml of distilled water.
2. 5 g PEG (MW 1000) was dissolved in 20 ml acetonitrile.
3. The PEG solution was added to the Mg/G dispersion over 15 minutes with stirring. The solvent was removed by rotary evaporation, and the product was oven dried.

Method 2: PEG at pH 8.0
1. 5 g PEG (MW 1000) was dissolved in 10 ml distilled water.
2. 1 g Mg/G was dispersed in distilled water (pH 7.8) and adjusted to pH 8.0 with 10 M potassium hydroxide.
3. PEG solution was added slowly. The mixture was left overnight stirring, centrifuged, washed, and dried.

PEG Coupling with MoF: M-PEG-3-OH (Axios Pharma) was activated using N, N-carbonyl diimidazole (Chem-Impex). 0.91 g (6.09 mmol) of the activator was added to M-PEG-3-OH in 30 ml dioxane, stirred at 37° C. for 2 hours, and evaporated. The activated PEG, characterized by an IR peak at 1720 cm⁻¹, was used for coupling.

NAD-MA-GA: Preparation of stable magnesium/gallic acid framework.

Step 1: Preparation of the Bivalent Cations (Mg, Zn, Ca).

Raw Materials
1. Magnesium chloride (anhydride) CAS #7786-30-3, lot #S8314333408 obtained from Sigma-Aldrich.
2. Gallic acid monohydrate, CAS #5996-86-8, lot #1003710701 from Sigma-Aldrich.
3. Distilled water obtained commercially.
4. Potassium Hydroxide (Acros Pharm)

A three-necked round-bottom flask equipped with a stirrer, thermometer, and condenser was charged with 250 ml of distilled water. The stirring started, and 11.40 g of gallic acid monohydrate (60.1 mm) was added. Stirring continued for 10 minutes.

Magnesium chloride 3 g (31.5 mmoles) was slowly added. Approximately 95% of the solids dissolved forming a beige solution. The pH of this solution was 3.8-4.0, and then adjusted to pH 8.0 at 25° C. with 10 M Potassium Hydroxide. The solution turned brownish in color and was clear. All the solids dissolved at this point, and the stirring continued while heating increased to 102° C., when refluxing started. The temperature was maintained at 102° C. for 24 hours. This solution was cooled and transferred into centrifuge tubes. It was centrifuged for thirty minutes at 4704 G Force (8000 rpm). Dupont Sorval CLC-28.

The top layer was decanted, and the tubes filled with the solids were washed with water (2×15 ml) and three times with ethanol. The solids were transferred to a China dish and dried at 75° C. for 6 hours. Yield 4.9 gms (˜78%).

Fourier-transform infrared (FTIR) Analysis (Perkin-Elmer Spectrum Two) confirmed the absence of Gallic Acid.

Step 2: A 500 mL three-neck round-bottom flask equipped with a stirrer, thermometer, and condenser was charged with 250 mL of distilled water. Gallic Acid 17.1 g [mmol] was slowly added and stirred for 10 minutes. 4.5 g magnesium chloride was added to this solution. This unique methodology was developed because of the current method's large volumes of water required. Mg/Ga was produced by converting gallic acid to its acid chloride (galloyl chloride) by treating it with Mg without solvents. After 3 hours of reflux, excess thionyl chloride evaporated, the product was washed with hexane, and finally with methanol.

The pH was adjusted to 8.0 with 10 M Potassium Hydroxide. Heating started and left overnight at 102-103° C. The next day it was cooled, centrifuged (Dupont Sorval CLC-28) at 8000 rpm. The top layer was decanted, and the precipitate was washed with 25 mL×2 water and 2×30 mL alcohol. The product was dried at 75° C. for three hours. Yield 9.0 g.

Step 3: Framework preparation of zinc/gallic acid.

To illustrate the framework, 8.71 g of Zinc chloride was charged into a 500 mL 3-neck flask. 250 ml of distilled water was added, followed by 11.4 g gallic acid monohydrate (0.12 M). Stirring continued for 10 minutes. The solution was observed to be slightly cloudy. The pH was adjusted to pH 10 using 10 M Potassium Hydroxide (˜40 mL). Initially, a precipitate formed but dissolved upon heating. The mixture was left overnight at 97-98° C., cooled, and filtered through a Buchner funnel. It was washed with water (3×50 mL) and Ethanol (2×50 ml), and the solids were dried under vacuum. Yield 24 g (80% of the product).

Step 4: Encapsulation of Mg/GA Framework with NAD

Raw Materials
1. Magnesium chloride (anhydride) CAS #7786-30-3, lot #S8314333408 obtained from Sigma-Aldrich.
2. Gallic acid monohydrate, CAS #5996-86-8, lot #1003710701 from Sigma-Aldrich.
3. NAD free acid 100% CAS #10127965001, Lot #71109186 from Sigma-Aldrich.
4. M-PEG3-Alcohol CAS #AP11237, Lot A59251 from Axios Pharma.
5. N-carbonyl diimidazole Lot #001545-140728 from Chem-Impex

PEG Coupling with MoF: M-PEG-3-OH was activated using N, N-carbonyl diimidazole. 0.91 g (6.09 mmol) of the activator was added to M-PEG-3-OH in 30 ml dioxane, stirred at 37° C. for 2 hours, and evaporated. The activated PEG, characterized by an IR peak at 1720 cm⁻¹, was used for NAD coupling.

NAD was encapsulated with Mg-GA by adding 66.34 mg of NAD dissolved in 150 mL of methanol under a soda-lime tube. 25 mg Mg/G was dispersed in 50 ml of ethanol and added to the NAD solution over 10-15 minutes. The mixture was stirred at 25° C. for 2 hours, centrifuged (8000 rpm, 25 minutes), washed, and dried. FTIR analysis indicated changes in the fingerprint region.

SOD-Mg—Ga: Preparation of stable magnesium/gallic acid framework.

Step 1: Preparation of the Bivalent Cations (Mg, Zn, Ca).

Raw Materials
1. Magnesium chloride (anhydride) CAS #7786-30-3, lot #S8314333408 obtained from Sigma-Aldrich.
2. Gallic acid monohydrate, CAS #5996-86-8, lot #1003710701 from Sigma-Aldrich.
3. Distilled water obtained commercially.
4. Potassium Hydroxide (Acros Pharm)

A three-necked round-bottom flask equipped with a stirrer, thermometer, and condenser was charged with 250 ml of distilled water. The stirring started, and 11.40 g of gallic acid monohydrate (60.1 mm) was added. Stirring continued for 10 minutes.

Magnesium chloride 3 g (31.5 mmoles) was slowly added. Approximately 95% of the solids dissolved forming a beige solution. The pH of this solution was 3.8-4.0, and then adjusted to pH 8.0 at 25° C. with 10 M Potassium Hydroxide. The solution turned brownish in color and was clear. All the solids dissolved at this point, and the stirring continued while heating increased to 102° C., when refluxing started. The temperature was maintained at 102° C. for 24 hours. This solution was cooled and transferred into centrifuge tubes. It was centrifuged for thirty minutes at 4704 G Force (8000 rpm). Dupont Sorval CLC-28.

The top layer was decanted, and the tubes filled with the solids were washed with water (2×15 ml) and three times with ethanol. The solids were transferred to a China dish and dried at 75° C. for 6 hours. Yield 4.9 gms (˜78%).

Fourier-transform infrared (FTIR) Analysis (Perkin-Elmer Spectrum Two) confirmed the absence of Gallic Acid.

Step 2: A 500 mL three-neck round-bottom flask equipped with a stirrer, thermometer, and condenser was charged with 250 mL of distilled water. Gallic Acid 17.1 g [mmol] was slowly added and stirred for 10 minutes. 4.5 g magnesium chloride was added to this solution. This unique methodology was developed because of the current method's large volumes of water required. Mg/Ga was produced by converting gallic acid to its acid chloride (galloyl chloride) by treating it with Mg without solvents. After 3 hours of reflux, excess thionyl chloride evaporated, the product was washed with hexane, and finally with methanol.

The pH was adjusted to 8.0 with 10 M Potassium Hydroxide. Heating started and left overnight at 102-103° C. The next day it was cooled, centrifuged (Dupont Sorval CLC-28) at 8000 rpm. The top layer was decanted, and the precipitate was washed with 25 mL×2 water and 2×30 mL alcohol. The product was dried at 75° C. for three hours. Yield 9.0 g.

Step 3: Framework preparation of zinc/gallic acid.

To illustrate the framework, 8.71 g of Zinc chloride was charged into a 500 mL 3-neck flask. 250 ml of distilled water was added, followed by 11.4 g gallic acid monohydrate (0.12 M). Stirring continued for 10 minutes. The solution was observed to be slightly cloudy. The pH was adjusted to pH 10 using 10 M Potassium Hydroxide (˜40 mL). Initially, a precipitate formed but dissolved upon heating. The mixture was left overnight at 97-98° C., cooled, and filtered through a Buchner funnel. It was washed with water (3×50 mL) and Ethanol (2×50 ml), and the solids were dried under vacuum. Yield 24 g (80% of the product).

Loading MgGA with SOD

STEP 1: Preparation

This illustrates the loading of SOD onto magnesium/gallic acid.

Raw materials:
1. Magnesium chloride (anhydride) CAS #7786-30-3, lot #S8314333408 obtained from Sigma-Aldrich.
2. Gallic acid monohydrate, CAS #5996-86-8, lot #1003710701 from Sigma-Aldrich.
3. SOD CAS #57571-30 KU, MW 32,000, Lot #0000288474 from Sigma-Aldrich.

STEP 2: Loading SOD into Mg-GA

SOD was encapsulated with Mg/GA by adding 3.2 mg of SOD dissolved in 5 ml distilled water. 10 ml of Mg/G dispersion was prepared and combined with the SOD solution over 5 minutes. Stirred at 37° C. for 2 hours, the solution turned brownish. After drying, 300 mg of Mg/G-SOD was obtained.

Pharmacokinetic Studies with (pegylated)NAD-Mg-GA MOF

The (pegylated)NAD-Mg-GA (as provided in FIG. 7 or FIG. 8) pharmacokinetic studies were performed by the PharmOptima laboratory in Portage, MI with MOF Science as sponsor. The Test article and dosing formulations were prepared as follows:
1. Identification: MG/G/NAD in Tris Buffered Saline
2. Alternate Identification: C₂₁H₂₇N₇₁O₄P₂.Mg₁ or Mg₂G
3. Batch/Lot No.: 5L291/24
4. Expiration/Retest Date: Feb. 1, 2027
5. Physical Description: Dark Purple
6. Storage Conditions: Refrigerated (2 to 8° C.)
7. Supplier: Sarchem Laboratories Inc.
8. Test Material Contact: Dr. Sam Kumar

Male BALB/c mice from Charles River Laboratories were used, totaling 39 mice, plus 8 spares, which will be used in the study with approximately 6 weeks of age at arrival.

The experimental design was developed with 4 distinct groups:
A. Group 1: Control group with 3 animals that were not dosed.
B. Group 2: Experimental group with 12 animals using 5 mg/kg at a concentration of 0.5 mL/mg of NAD-Mg-GA.
C. Group 3: Experimental group with 12 animals using 10 mg/kg at a concentration of 1 mL/mg of NAD-Mg-GA.
D. Group 4: Experimental group with 12 animals using 20 mg/kg at a concentration of 2 mL/mg of NAD-Mg-GA.

Animals in Groups 2-4 received concentrations of test article at a volume of 10 mL/kg.

The doses were administered orally by gavage.

The oral route was selected as the intended route of administration in humans. The chosen dose levels selected by the Sponsor were designed to achieve measurable concentrations of the test article in the blood plasma at all levels. None of the chosen doses presented toxicity levels.

Blood collection timepoints were performed at the following time periods:
A. Group 1—4 h and 24 h
B. Groups 2-4—0.25 h, 0.5 h, 1 h. 2 h, 4 h, 8 h and 24 h.

0.2-0.3 mL was collected from 3 animals per group at each collection point. Each animal was bled at 2 collection points. After the second blood collection, the animals were euthanized according to the following protocol.

Tissue Collection and Homogenization

Immediately after blood collection, 2, 4, 8 and 24 hours post-dose, 3 animals from each group, in groups 2 to 4, were euthanized by CO2 inhalation. Animals in Group 1 were euthanized 24 hours post-dose. Brains were collected, placed in PRESCELLY® tubes, weighed, placed on dry ice and stored at −80° C. for further processing and analysis. PharmOptima analyzed brain and plasma for NAD and magnesium gallate concentrations using LC MS/MS methods developed by PharmOptima.

Pharmacokinetic analyses were performed using standard WinNonlin non-compartmental methods (Tmax, Cmax, Tlast, AUClast) from composite concentration-time data generated for plasma and brain samples. Pharmacokinetic analyses were conducted by Part 58 Consulting LLC, and pharmacokinetic data were submitted to the sponsor.

Study Results

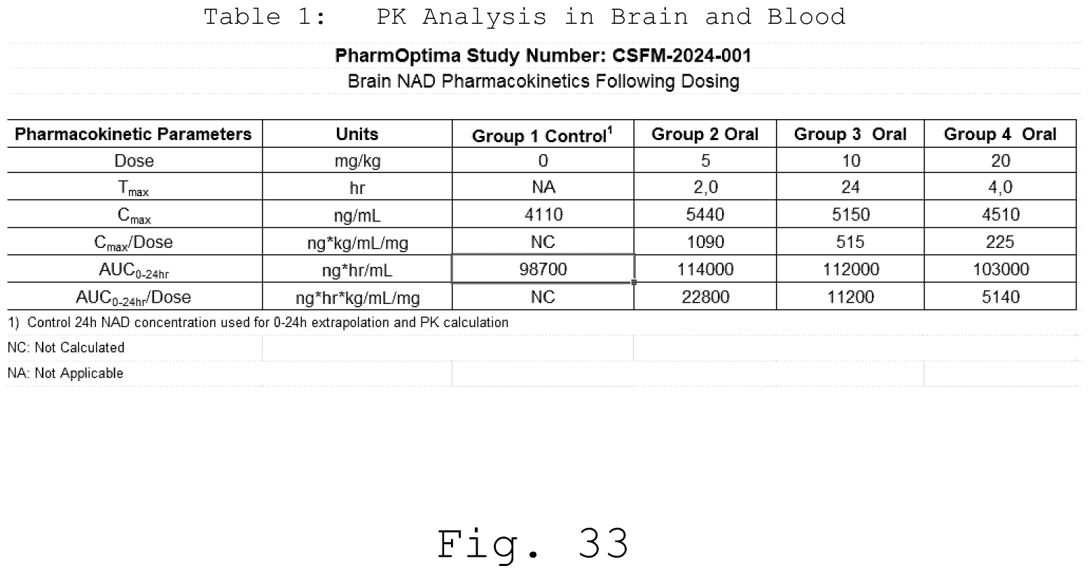

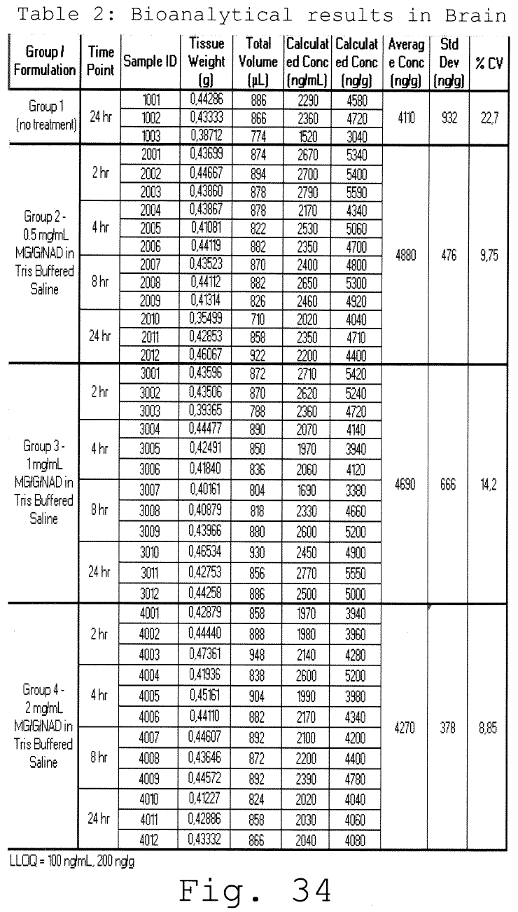

The results of the pharmacokinetic studies are provided in the Table 1, see FIG. 33 and Table 2, see FIG. 34. Table 1 provides data related to the PK analysis in brain and blood. Table 2 provides data related to bioanalytical results in the brain.

The Cmax results in group 2 were more expressive, demonstrating a 32.4% increase in NAD concentrations when compared to the control group (Table 1).

In groups 3 and 4, the study demonstrated that the higher the dose, the lower the NAD concentrations in the nervous tissue, with 25.3% and 9.5%, respectively (Table 1).

These results demonstrated that the 5 mg/kg dose was the one that presented the best result (Table 1 and 2).

Regarding NAD concentrations in the bloodstream, the results followed the same pattern but with less NAD present compared to the tissues (Table 1).

The AUC0-24 h results were 15.6% in group 2, followed by 13.5% in group 3 and 4.4% in group 3 (Table 1).

The difference in the PK test of the greater presence of NAD inside the cells compared to the bloodstream can be explained by the fact that MOFs are disassembled at a pH close to 7, as occurs in the cytoplasm of neurons. Since blood plasma has a pH of 7.4, and the pH follows a logarithmic scale, 0.4 is four times more acidic, which favors the disassembly of magnesium gallate, releasing NAD more efficiently inside the cells. Knowing that biological membranes, in general, are a challenging barrier to the metabolization of any exogenous ingredient, having more NAD in the tissues than in the blood plasma is a promising way to replace this coenzyme.

Not only can the presence of NAD in the tissues help in numerous pathologies, but gallate, when dissociated from magnesium, will form gallic acid, a potent activator of endogenous antioxidant mechanisms, even favoring the metabolism of NAD itself.

Observing the averages of the results presented in brain tissue, group 2, that is, IP, demonstrated less presence of NAD in the brain than group 1. (Pegylated) NAD-Mg-GA presented 5.5% more NAD in the brain than group 2.

In the liver, t there was no significant difference between groups 1 and 2.

In muscle tissue, the result was the most expressive among all groups in this study. Group 1 had 59% more NAD than group 2.

Finally, in the heart, group 2 presented 11% more NAD levels when compared to group 1.

This variation in the presence of NAD can be explained by some determining factors, among them the pH of the cytoplasm of each tissue. Since skeletal muscle has a more acidic pH than the other tissues present in this study, the (pegylated) NAD-Mg-GA structure causes MOF to move more efficiently to a more acidic environment, dismantling the molecule and releasing NAD. Magnesium acts as a taxi driver for the molecule, seeking out more acidic environments, which leads us to believe that this type of carrier is the best way to increase NAD levels in tissues that experience pH changes, as occurs with several diseases, since diseases are a reflection of inflammatory processes and inflammation lowers pH, including neurodegenerative diseases, cancer, diabetes, hepatic steatosis, rheumatoid arthritis, among others. The drop in mitochondrial membrane potential in diseases and also in aging generates a more acidic pH in the cytoplasm, which favors the use of MOF to recover NAD levels within the tissues.

In conclusion, the first study indicated a 32.4% increase in NAD in the brain tissue in the group that used (pegylated)NAD-Mg-GA when compared to the control group that did not receive NAD-Mg-GA. Based on the second study, when administered orally, (pegylated)NAD-Mg-GA presented superior results to injectable NAD when evaluated within the tissues. These results are due to two factors. First, MOF acts as a taxi driver of molecules to the interior of the tissues, dismantling their structure at a pH slightly more acidic than that of blood plasma, where it preserves the NAD within its structure until its total disintegration. Second, the more acidic the tissue environment, the better the bioavailability of (pegylated)NAD-Mg-GA. This can be explained by the fact that muscle tissue has a more acidic pH than other tissues, providing a superior result (59% more than injectable NAD in muscles) when compared to other tissues (5.5% more than injectable NAD in the brain, for example). Since the second study evaluated the tissues of healthy rats, we can conclude that in diseases and metabolic disorders, the pH is more acidic than physiological within the tissues, which further helps direct NAD-Mg-GA, improving the bioavailability of NAD for metabolic correction. This would be the main focus of MOF. Having bioavailability in the bloodstream may be important. However, having better bioavailability within the tissues makes more of a difference for a therapeutic response.

Accordingly, (pegylated)NAD-Mg-GA proved to be more effective when seeking to increase NAD levels in tissues that are inflamed, as in the case of aging and associated diseases. This may change the direction of NAD administration, which does not have intestinal receptors for oral use. Our study demonstrated that (pegylated)NAD-Mg-GA is more effective than injectable NAD and can be prescribed by physicians as NAD replacement therapy without needing to be administered intravenously (IV) or even intramuscularly (IM). This allows for greater overall patient comfort.

As NAD plays a role in various diseases, syndromes, and disorders, the embodiments of the invention may be used in administering NAD to individuals in need, such those suffering from metabolic syndrome (including type 2 diabetes, fatty liver, heart disease (including high blood pressure, heart failure, cardiovascular inflammation), stroke and inflammatory disorders), fibromyalgia, rheumatoid arthritis, neurological diseases (such as Alzheimer's disease, Parkinson's disease and cognitive deficits in general (memory and focus)), chronic fatigue, sleep disorders, and controlling aging.

Referring to FIGS. 19-32, alternative examples of loading molecules are provided. Such examples are provided for illustrative purposes and are not meant to be limiting in scope. Each of the embodiments described in FIGS. 21-35 may have any structure or feature as described for any of the embodiments described in FIGS. 5-20.

FIG. 19 illustrates a quercetin-loaded MG-Ga MOF. Quercetin-loading magnesium-gallate has specific mitochondrial biological functions. When this molecule enters cells, it disintegrates into magnesium, quercetin and gallic acid. Quercetin is a flavonoid that has a direct effect on proteins that act on mitochondrial biogenesis, such as peroxisome proliferator-activated receptor-γ coactivator 1-α (PGC-1a) and AMP kinase (AMPK). Quercetin improves lipid and glucose metabolism, improving insulin sensitivity, and modulating gut microbiota. Quercetin also modulates inflammatory processes as well as oxidative stress in cells.

In certain embodiments, the quercetin-loaded MG-Ga MOF may be used to deliver quercetin, MG, Ga to individuals in need thereof, such those suffering from metabolic diseases, such as Type 2 diabetes, heart disease, including heart failure, high blood pressure and cardiovascular inflammation, and chronic fatigue

FIG. 20 illustrates a Ursolic Acid-loaded MG-Ga MOF. When this molecule enters cells, it disintegrates into magnesium, ursolic acid and gallic acid. Usolic acid in liposomes (Merotaine) are known to increase the ceramide content in human skin over 11 days. Ursolic acid increases the amount of brown fat tissue while decreasing white fat tissue in mammalian metabolism, reducing the percentage of body fat. Ursolic acid can also act on irisin, signaling an anabolic response to muscle tissue even without physical activity.

In certain embodiments, the Ursolic Acid-loaded MG-Ga MOF may be used to deliver Ursolic acid to individuals in need, such as those suffering from metabolic disease, such as Type 2 diabetes, cardiovascular inflammation, vascular inflammation, and sarcopenia

FIG. 21 an illustrates an Alpha Lipoic Acid-loaded MG-Ga MOF. When this molecule enters cells, it disintegrates into magnesium, glutathione and gallic acid. Alpha-lipoic acid works as an important cofactor for many enzyme complexes including mitochondrial respiratory enzymes. Alpha lipoic acid plays a crucial role in mitochondrial dehydrogenases, acting as one of the essential cofactors in the production of ATP. Alpha lipoic acid also modulates inflammatory processes as well as oxidative stress in cells.

In certain embodiments, the Alpha Lipoic Acid-loaded MG-Ga MOF may be used to deliver Alpha Lipoic Acid to individuals in need thereof, such as those suffering from metabolic syndrome disease, including Type 2 diabetes, heart disease, including heart failure, high blood pressure and cardiovascular inflammation, liver dysfunction, including hepatic steatosis and liver cirrhosis, chronic fatigue, and neurological disease, such as to improve memory and focus.

FIG. 22 is an illustrative example of a pyrroloquinoline quinone (PQQ)-loaded MG-Ga MOF. When this molecule enters cells, it disintegrates into magnesium, PQQ and gallic acid. Pyrroloquinoline quinone (PQQ) influences energy-related metabolism and neurologic functions in animals. The mechanism of action involves interactions with cell signaling pathways and mitochondrial function. One of the most impressive qualities of PQQ in humans is its antioxidant effects. It's about 100 times as powerful as vitamin C and increases Nrf2, a critical pathway that increases endogenous antioxidant production in cells. PQQ can also activate the PGC-1a gene, sparking mitochondrial biogenesis, and promoting mitochondrial growth even in non-young tissues.

In certain embodiments, the pyrroloquinoline quinone (PQQ)-loaded MG-Ga MOF may be used to deliver PQQ to individuals in need thereof, such as those suffering from metabolic syndrome disease, including Type 2 diabetes, heart disease, including heart failure, high pressure blood and cardiovascular inflammation, liver dysfunction, including hepatic steatosis and liver cirrhosis, chronic fatigue, and neurological disease, such as to improve memory and focus.

FIG. 23 is an illustrative example a ubiquinol/CoQ10-loaded MG-Ga MOF. When this molecule enters cells, it disintegrates into magnesium, ubiquinol and gallic acid. Ubiquinol is a coenzyme that acts at the center of electron transport in the mitochondrial respiratory chain, more specifically in complex III. It bridges the gap between the electrons donated by NADH and oxygen. ATP production in cells depends on ubiquinol.

In certain embodiments, ubiquinol/CoQ10-loaded MG-Ga MOF may be used to deliver ubiquinol to an individual in need thereof, such as cardiovascular disease such as heart failure, high blood pressure and cardiovascular inflammation, chronic fatigue, or fertility

FIG. 24 is an illustrative example of a Fisetin-loaded MG-Ga MOF. When this molecule enters cells, it disintegrates into magnesium, fisetin and gallic acid. Fisetin is a flavonoid with direct influence on several metabolic processes, including strengthening antioxidant defenses, regulating lipid metabolism and modulating mitochondrial function. Fisetin plays a role in regulating amino acid and metabolism energy homeostasis, impacting cellular senescence and potentially acting as a senotherapeutic agent helping to control cellular aging and associated diseases.

In certain embodiments, Fisetin-loaded MG-Ga MOF may be used to deliver fisetin to an individual in need thereof, such as metabolic syndrome diseases, such as Type 2 diabetes, heart disease, including heart failure, high blood pressure and cardiovascular inflammation, liver dysfunction, including hepatic steatosis and liver cirrhosis, chronic fatigue, and neurological disease, such as Alzheimer's disease, and improving memory and focus.

FIG. 25 is an illustrative example of a glutathione-loaded MG-Ga MOF. When this molecule enters cells, it disintegrates into magnesium, glutathione and gallic acid. Glutathione is a tripeptide with antioxidant action that removes hydrogen peroxide by converting it into water and reducing the risk of ROS produced in cells. Glutathione acts in the mechanisms of elimination of heavy metals, being a crucial endogenous component for detoxification mechanisms. Riboflavin-5-phosphate is the coenzyme of glutathione reductase in the conversion of oxidized glutathione into reduced glutathione. R5P also acts in the reduction of NAD, converting it into NADH, that is, its reduced form, keeping metabolism active. Selenium acts as an enzymatic cofactor for the enzyme glutathione peroxidase, which converts reduced glutathione into oxidized glutathione. In this process, glutathione removes free radicals and heavy metals from the metabolism. The action of selenium with R5P can keep the glutathione loop acting for longer in the metabolism by activating the enzymes glutathione peroxidase and glutathione reductase, respectively.

In certain embodiments, glutathione (R5P-Se)-loaded MG-Ga MOF may be used to deliver glutathione to an individual in need thereof, such as liver diseases, such as liver dysfunction, including hepatic steatosis and liver cirrhosis, chronic fatigue, chronic fatigue, and chronic inflammatory processes and altered immunity.

FIG. 26 is an illustrative example of a SOD1/SOD2 (80/20)-loaded MG-Ga MOF. When this molecule enters cells, it disintegrates into magnesium, SOD (1 & 2) and gallic acid. SOD 1 and SOD2 are two types of SOD, cytoplasmic and mitochondrial respectively. Both have the same action in metabolism but in different proportions present in cells 8:2 respectively.

In certain embodiments, SOD1/SOD2-loaded MG-Ga MOF may be used to deliver SOD1 and SOD2 to an individual in need thereof, such as those suffering from metabolic syndrome diseases, such as Type 2 diabetes, heart disease, including heart failure, high blood pressure and cardiovascular inflammation, liver dysfunction, including hepatic steatosis and liver cirrhosis, chronic fatigue, and neurological diseases, such as Parkinson's disease, or Amyotrophic lateral sclerosis

FIG. 27 an illustrative example an astaxanthin-loaded MG-Ga MOF. When this molecule enters cells, it disintegrates into magnesium, astaxanthin and gallic acid. Astaxanthin is a carotenoid that plays a crucial role in mitochondrial membrane potential. This carotenoid has the ability to increase membrane potential, especially in complex IV of the respiratory chain, improving electron transport and, therefore, ATP synthesis. This action of astaxanthin makes this carotenoid an important protector against UV radiation. Astaxanthin has also been shown to be effective in signaling proteins that act in mitochondrial biogenesis. Studies have shown that astaxanthin can be used to protect against pathologies resulting from aging.

In certain embodiments, astaxanthin-loaded MG-Ga MOF may be used to deliver astaxanthin to an individual in need thereof, such as those suffering from metabolic syndrome diseases, such as Type 2 diabetes, heart disease, including heart failure, high blood pressure and cardiovascular inflammation, liver dysfunction, including hepatic steatosis and liver cirrhosis, chronic fatigue, neurological diseases, such as Alzheimer's disease, and memory and focus, macular degeneration and eczema.

FIG. 28 an illustrative example of a Beta-caryophellene-loaded MG-Ga MOF. When this molecule enters cells, it disintegrates into magnesium, beta-caryophyllene and gallic acid. Beta-caryophyllene is a terpenoid with anti-inflammatory action. It acts as an agonist of PPARa receptors, controlling the expression of NF-KB. Considered an analogue of cannabinoids, this terpenoid has an action on the central nervous system, helping to control inflammation.

In certain embodiments, Beta-caryophellene-loaded MG-Ga MOF may be used to deliver Beta-caryophellene to an individual in need thereof, such as those suffering from metabolic syndrome diseases, such as Type 2 diabetes, heart disease, including heart failure, high blood pressure and cardiovascular inflammation, liver dysfunction, including hepatic steatosis and liver cirrhosis, chronic fatigue, neurological diseases, such as Alzheimer's disease, and memory and focus.

FIG. 29 is an illustrative example of a berberine-loaded MG-Ga MOF. When this molecule enters cells, it disintegrates into magnesium, berberine and gallic acid. Berberine plays a significant role in several metabolic processes, primarily impacting glucose and lipid metabolism. It improves insulin sensitivity, regulates blood sugar, and potentially aids in weight control.

In certain embodiments, berberine-loaded MG-Ga MOF. may be used to deliver berberine to an individual in need thereof, such as those suffering from metabolic syndrome diseases, such as Type 2 diabetes, and neurological diseases, such as Alzheimer's disease, and memory and focus.

FIG. 30 is an illustrative example of a testosterone-loaded MG-Ga MOF. When this molecule enters cells, it disintegrates into magnesium, testosterone and gallic acid. Testosterone plays a crucial role in the metabolism of carbohydrates, fats and proteins. It affects muscle mass, body fat distribution and insulin sensitivity, all of which are important factors for metabolic health. Testosterone is also associated with behavior, since its levels, when within physiological standards, improve motivation. Its action on skeletal muscles and bones is crucial for preserving muscle mass and bone mass, especially in the elderly.

In certain embodiments, testosterone-loaded MG-Ga MOF. may be used to deliver testosterone to an individual in need thereof, such as those suffering from metabolic syndrome diseases, such as Type 2 diabetes or chronic fatigue, and neurological diseases, such as Alzheimer's disease, and memory and focus.

FIG. 31 is an illustrative example of a resveratrol-loaded MG-Ga MOF. When this molecule enters cells, it disintegrates into magnesium, resveratrol and gallic acid. Resveratrol is a polyphenol with significant action on cellular aging. Studies have revealed its action on sirtuins, especially sirtuins 1 and 3, the latter of which has mitochondrial action and may be an ally in NAD therapies. Resveratrol also has anti-inflammatory action, improving insulin sensitivity and controlling glucose metabolism.

In certain embodiments, resveratrol-loaded MG-Ga MOF may be used to deliver resveratrol to an individual in need thereof, such as those suffering from metabolic syndrome diseases, such as Type 2 diabetes, cardiovascular inflammation, pr vascular inflammation.

FIG. 32 is an illustrative example of a carnosine-loaded MG-Ga MOF. When this molecule enters cells, it disintegrates into magnesium, carnosine and gallic acid. Carnosine is a dipeptide formed by the amino acids beta-alanine and histidine. Its action as a pH buffer in myocytes makes this dipeptide a crucial ingredient for the entire skeletal muscle system, protecting against fatigue, exhaustion and injuries.

In certain embodiments, carnosine-loaded MG-Ga MOF may be used to deliver carnosine to an individual in need thereof, such as those suffering from metabolic syndrome diseases, such as Type 2 diabetes, chronic fatigue and sarcopenia.

All patents and publications mentioned in this specification are indicative of the levels of those skilled in the art to which the invention pertains.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A modified metal-organic framework (MOF) comprising:
A metal-organic framework comprising a magnesium-gallate (Mg-GA) network structure; and
a phosphate-functionalizing constituent.

2. The modified metal-organic frameworks (MOF) according to claim 1, wherein said phosphate-functionalizing constituent comprises one or more PEGylates (polyethylene glycol) with phosphate groups.

3. The modified metal-organic frameworks (MOF) according to claim 1, further including a loading molecule.

4. The modified metal-organic frameworks (MOF) according to claim 3, wherein said loading molecule is a coenzymes, enzymes, flavonoids, carotenoids, hormones, or phytochemicals.

5. The modified metal-organic frameworks (MOF) according to claim 3, wherein said loading molecule is NAD, SOD, quercetin, ursolic acid, alpha lipoic acid, pyrroloquinoline quinone (PQQ), ubiquinol/CoQ10, fisetin, glutathione, SOD1/SOD2, astaxanthin, beta-caryophellene, berberine, testosterone, resveratrol, or carnosine.

6. The modified metal-organic frameworks (MOF) according to claim 3, wherein said loading molecule is NAD.

7. The modified metal-organic frameworks (MOF) according to claim 6, wherein said NAD is loaded with one or more PEGylates (polyethylene glycol) with phosphate groups.

8. The modified metal-organic frameworks (MOF) according to claim 3, wherein said loading molecule is SOD.

9. The modified metal-organic frameworks (MOF) according to claim 1, wherein said MOF is formed as a three-dimensional structure.

10. The modified metal-organic frameworks (MOF) according to claim 1, wherein said MOF is formed as a three-dimensional structure having micropores.

11. A modified metal-organic framework (MOF) loaded with a coenzyme comprising:
a metal-organic framework comprising a magnesium-gallate (Mg-GA) network structure;
Nicotinamide Adenine Dinucleotide (NAD);
and a phosphate-functionalizing constituent.

12. The modified metal-organic frameworks (MOF) according to claim 11, wherein said phosphate-functionalizing constituent comprises one or more PEGylates (polyethylene glycol) with phosphate groups.

13. The modified metal-organic frameworks (MOF) according to claim 11, wherein said phosphate-functionalizing constituent is loaded to said NAD.

14. The modified metal-organic frameworks (MOF) according to claim 11, wherein said MOF is formed as a three-dimensional structure.

15. The modified metal-organic frameworks (MOF) according to claim 11, wherein said MOF is formed as a three-dimensional structure having micropores.

16. A method of delivering NAD comprising:
Administering to an individual in need therefore, a modified metal-organic framework (MOF) loaded with NAD, said modified metal-organic framework (MOF) comprising:
a metal-organic framework comprising a magnesium-gallate (Mg-GA) network structure;
Nicotinamide Adenine Dinucleotide (NAD); and
a phosphate-functionalizing constituent.

17. The method according to claim 16, wherein said modified metal-organic framework (MOF) loaded with a coenzyme is administered orally.

18. The method according to claim 16, wherein said phosphate-functionalizing constituent comprises one or more PEGylates (polyethylene glycol) with phosphate groups.

19. The method according to claim 16, wherein said phosphate-functionalizing constituent is loaded to said NAD.

20. The method according to claim 16, wherein said individual is in need of a treatment for a metabolic syndrome disease, a neurological disease, or aging process.

21. The method according to claim 16, wherein said modified metal-organic framework (MOF) is administered to an individual in need of NAD replacement therapy.

22. A modified metal-organic framework (MOF) loaded with a coenzyme comprising:
a metal-organic framework comprising a magnesium-gallate (Mg-GA) network structure;
superoxide dismutase (SOD)

23. The modified metal-organic frameworks (MOF) according to claim 22, wherein said MOF is formed as a three-dimensional structure.

24. The modified metal-organic frameworks (MOF) according to claim 22, wherein said MOF is formed as a three-dimensional structure having micropores.

25. A method of delivering SOD to an individual comprising: administering to an individual in need therefore, a modified metal-organic framework (MOF) loaded with SOD, said modified metal-organic framework (MOF) comprising:
a metal-organic framework comprising a magnesium-gallate (Mg-GA) network structure; and
superoxide dismutase (SOD).

26. The method according to claim 25, wherein said modified metal-organic framework (MOF) loaded with SOD is administered orally.

27. The method according to claim 25, wherein said individual is suffering from a metabolic syndrome disease, a neurological disease, aging process.
